(19) United States
(12) Patent Application Publication
(10) Pub. No.: US 2016/0060688 A1
(43) Pub. Date: Mar. 3, 2016

(54) USE OF PROBES FOR MASS SPECTROMETRIC IDENTIFICATION OF MICROORGANISMS OR CELLS AND ASSOCIATED CONDITIONS OF INTEREST

(71) Applicant: ADVANDX, INC., Woburn, MA (US)

(72) Inventors: James M. Coull, Westford, MA (US); Martin Fuchs, Uxbridge, MA (US); Mark J. Fiandaca, Princeton, MA (US); Alisha Perelta, Burlington, MA (US); Jan Trnovsky, Saugus, MA (US)

(73) Assignee: ADVANDX, INC., Woburn, MA (US)

(21) Appl. No.: 14/735,304

(22) Filed: Jun. 10, 2015

Related U.S. Application Data
(63) Continuation of application No. PCT/US2013/074026, filed on Dec. 10, 2013.

(51) Int. Cl. C12Q 1/68

(52) U.S. Cl. CPC C12Q 1/6841

(57) ABSTRACT

This invention pertains to identifying one or more hybridization probes sequestered within (or optionally released from the intact) cells or microorganisms by mass spectrometry to thereby determine a trait of the cells or microorganisms and/or to identify the cells or microorganisms themselves. The cells or microorganisms can come from a subject and the information obtained from the mass spectrometry analysis may, if clinically relevant, optionally be used to diagnose and/or treat the subject.

### CROSS REFERENCE TO RELATED APPLICATIONS

This application claims the benefit of International Patent Application No. PCT/US2013/074026, filed on Dec. 10, 2013; which claims priority to U.S. Provisional Patent Application No. 61/735,410, filed on Dec. 10, 2012. The entire contents of each of the foregoing applications are hereby incorporated herein by reference.

The section headings used herein are for organizational purposes only and should not be construed as limiting the subject matter described in any way.

### BRIEF DESCRIPTION OF DRAWINGS

The skilled artisan will understand that the drawings, described below, are for illustration purposes only. The drawings are not intended to limit the scope of the present teaching in any way.

FIG. 1: Seven superimposed MALDI spectra (restricted to the 4000 to 4700 m/z range) for seven species of bacteria grown in simulated blood cultures and individually detected with a probe cocktail comprising eight PNA probes (seven species-specific, one universal).

FIG. 2: Nine superimposed MALDI spectra (restricted to the 4000 to 4700 m/z range) for nine positive blood cultures obtained from a clinical microbiology lab individually detected with a probe cocktail comprising eight PNA probes (seven species-specific, one universal).

FIG. 3: Two superimposed MALDI spectra (restricted to the 4000 to 4700 m/z range) for two urine cultures obtained from a clinical microbiology lab individually detected with a probe cocktail comprising eight PNA probes (seven species-specific, one universal).

FIG. 4: Five superimposed MALDI spectra (restricted to the 4000 to 4700 m/z range) for two simulated blood cultures spiked with either S. aureus or S. epidermidis in different ratios individually detected with a probe cocktail comprising eight PNA probes (seven species-specific, one universal).

FIG. 5: Five superimposed MALDI spectra (restricted to the 4000 to 4700 m/z range) for five species of bacteria grown in simulated blood culture and individually detected with a probe cocktail comprising eight PNA probes (seven species-specific, one universal) and processed using a Smart Wash.

FIG. 6: Two microscopic images for two species of bacteria grown in blood culture, and each individually detected with a probe cocktail comprising one species specific PNA probe labeled with fluorescein. Images are presented in the negative.

All literature and similar materials cited in this application, including but not limited to patents, patent applications, articles, books and treatises, regardless of the format of such literature or similar material, are expressly incorporated by reference herein in their entirety for any and all purposes.

### DESCRIPTION

### 1. Field

This invention pertains to the field of determining microorganisms or cells using mass spectrometry, including but not limited to, the determination of antibiotic resistance strains of bacteria.

### 2. Introduction

Pure colonies and liquid cultures of microorganisms can be identified using mass spectrometry (MS), particularly by use of matrix assisted laser desorption ionization-time of flight (MALDI-TOF) mass spectrometers. As a result, mass spectrometers may become a central instrument platform within microbiology laboratories. However, because accurate identification currently requires that the organisms first be isolated as pure cultures or colonies and made essentially free of contaminating media and/or other matter such as patient material (blood, etc.), there is a significant delay between when a sample is first obtained and when the accurate identification by mass spectrometry (MS) can be made. Often this delay to obtain a pure isolate can be many hours or days, and in the case of clinical microbiology where rapid identification results are required to effectively treat patients (i.e., proper administration of antimicrobial drugs), such delays are associated with unsatisfactory patient outcomes, increased healthcare costs and misusage of antibiotics.

Blood culture is a standard specimen (i.e., sample) type that is commonly received for analysis in the clinical microbiology laboratory. When a blood culture turns positive, indicating that an organism is present within the culture, the culture is plated to isolate the organism as a single clonal colony in order for the MS to provide an accurate identification result. Despite efforts to process blood cultures directly to concentrate and purify microorganisms without the need for colony isolation, the accuracy is typically only in the range of 60-80%. In contrast, after clonal isolation by plating, the accuracy is in the range of greater than 95%. As such, there is a need to improve the accuracy of MS identification results directly from blood cultures.

Currently, the identification of microorganisms by MS is performed by comparing the masses (i.e., peak position and intensities that correlate with mass to charge (m/z) ratios) observed in the mass spectrum of the unknown sample to a database of mass spectra collected from known organisms. The majority of the mass peaks in these mass spectra represent the highly abundant ribosomal proteins which vary uniquely in mass between each species and genus of organism.

The determination of drug resistance or sensitivity is another important activity of the clinical microbiology laboratory. Typically, drug resistance and/or susceptibility of microorganisms are often determined using pure isolates in combination with phenotypic methods such microbroth dilution and disk diffusion. These properties may also be determined by use of automated phenotypic readers such as the VITEK® instrument sold by bioMerieux. In the former methods, the microorganism is exposed to a drug compound in a liquid solution and/or on a plate and the ability of the organism to grow is measured as a function of the drug presence and/or its concentration. In cases where a unique molecular mechanism is known, for example, methicillin resistant Staphylococcus aureus (MRSA), genotypic or protein content can also be identified/measured to make a determination. For MRSA, the genotypic methods are often PCR-based and involve the amplification of the mecA gene, the presence of which is highly correlated to a methicillin resistant phenotype. Another molecular method is to perform fluorescence in-situ hybridization (FISH) using PNA probes directed to the mecA messenger RNA (mRNA).

In the mecA PNA FISH assay a fluorescent signal demonstrates that a transcriptionally active mecA gene exists, which also correlates highly with the MRSA phenotype. The ultimate expression product responsible for the majority of MRSA is the PBP2a protein encoded by the mecA gene. Various antibody tests have been developed which utilize this protein as their target. While correlating very highly with the MRSA phenotype, the PBP2a protein cannot be detected directly from pure colonies or blood cultures with a high degree of accuracy by MS. This is most likely due to the low cellular abundance of PBP2a when compared to the ribosomal proteins. The high incidence of the ribosomal proteins makes it difficult to detect the PBP2a protein directly without additional purification steps and/or without substantially increasing the amount of sample that must be processed for the protein to be detectable.

There are additional resistance phenotypes or toxigenic phenotypes where specific proteins, genes or gene mutations are responsible for the resistance or toxigenicity phenotype of a microorganism. The genes and proteins can also be detected using antibodies or genotyping but most will likely remain refractory to determination by MS. These include, but are not limited to, the vanA and vanB gene products responsible for vancomycin resistance in enterococci, the toxin A and toxin B gene products associated with C. difficile caused diarrhea, and the carbapenemase gene products (VIM, VIP, NMD, OXA, etc.) associated with carbapenem drug resistance in gram negative bacilli. Resistance and toxigenicity are also often referred to as ‘traits’ of a microorganism.

### DEFINITIONS

For the purposes of interpreting of this specification, the following definitions will apply and whenever appropriate, terms used in the singular will also include the plural and vice versa. In the event that any definition set forth below conflicts with the usage of that word in any other document, the definition set forth below shall always control for purposes of interpreting the scope and intent of this specification and its associated claims. Notwithstanding the foregoing, the scope and meaning of any document incorporated herein by reference should not be altered by the definition presented below. Rather, said incorporated document should be interpreted as it would be by the ordinary practitioner based on its content and disclosure with reference to the content of the description provided herein.

The use of “or” means “and/or” unless stated otherwise or where the use of “and/or” is clearly inappropriate. The use of “a” means “one or more” unless stated otherwise or where the use of “one or more” is clearly inappropriate. The use of “comprise,” “comprises,” “comprising” “include,” “includes,” and “including” are interchangeable and not intended to be limiting. Furthermore, where the description of one or more embodiments uses the term “comprising,” those skilled in the art would understand that in some specific instances, the embodiment or embodiments can be alternatively described using language “consisting essentially of” and/or “consisting of.”

As used herein, the terms “administered” and “subjected” are interchangeable with respect to the treatment of a disease or disorder and both terms refer to a subject being treated with an effective dose of pharmaceutical composition comprising a compound disclosed herein by methods of administration such as parenteral or systemic administration.

As used herein, an “agent” is a chemical molecule of synthetic or biological origin. In the context of the present invention, an agent can be a molecule that can be used in a pharmaceutical composition. In some embodiments, the agent can be an antibiotic agent or agents. In some embodiments, the agent can provide a prophylactic or therapeutic value. In some embodiments, the small molecule compounds may (or may not) further comprise a pharmaceutically acceptable carrier.

As used herein an “aptamer” refers to a nucleic acid species that has been engineered through repeated rounds of in vitro selection or equivalently, SELEX (systematic evolution of ligands by exponential enrichment) to bind to various ‘molecular targets’ (not a nucleic acid target or target as defined below) such as a target nucleic acid.

As used herein, “chimera” refers to an oligomer comprising subunits of two or more different classes of subunits. For example, a chimera can comprise subunits of deoxyribonucleic acid (DNA) and locked nucleic acid (LNA) to be a DNA-LNA chimera, can comprise subunits of DNA and ribonucleic acid (RNA) to be a DNA-RNA chimers, can comprise subunits of DNA and peptide nucleic acid (PNA) to be a DNA-PNA Chimera, can comprise subunits of DNA, LNA and PNA (to be a DNA-LNA-PNA chimera) or can comprise subunits of RNA and LNA (to be a RNA-LNA chimera), etc. For example, an oligomer comprising both PNA and nucleic acid (DNA or RNA) subunits would be a PNA-DNA chimera or PNA-RNA chimera, either of which can just be referred to as a PNA chimera. It is to be understood that what the literature refers to as LNA probes are typically chimeras (according to this definition), since said “LNA probes” usually incorporate only one or a few LNA nucleotides into an oligomer that is primarily comprises of DNA or RNA subunits.

As used herein, “determining” refers to making a decision based on investigation, data, reasoning and/or calculation. Some examples of determining include detecting, identifying and/or locating (bacteria and/or traits) as appropriate based on the context/usage of the term herein.

As used herein, “diagnose” or “diagnosis” refers to recognizing a disorder, disease state or illness in a subject.

As used herein, “diagnostic” refers to methods of, or that yield, a diagnosis of a subject.

As used herein, the terms “disorder” and “disease” are used interchangeably and refer to any alteration in the state of the body or of some of its organs, interrupting or disturbing the performance of the functions and/or causing symptoms such as discomfort, dysfunction, distress, or even death to the person afflicted or those in contact with the person. A disease or disorder can also relate to distemper, ailing, ailment, malady, disorder, sickness, illness, complaint, indisposition, affection or infection.

As used herein the term “effective amount” refers to the amount of at least one therapeutic agent (e.g., small molecule compound) or pharmaceutical composition (e.g., a formulation) that can be administered to reduce or stop at least one symptom or condition of abnormal proliferation in a subject. For example, an effective amount may be considered as the amount sufficient to reduce a symptom or condition of the abnormal proliferation by at least 10%. An effective amount as used herein may also include an amount sufficient to prevent or delay the development of a symptom or condition of the disease, alter the course of a symptom or condition of disease (for example but not limited to, slow the progression of a symptom of the disease), or reverse a symptom or condition of the disease (e.g., an infection). Accordingly, the term “effective amount” or “therapeutically effective amount” refers to the amount of therapeutic agent needed to alleviate at least some of the symptoms or conditions experienced by a subject.

As used herein, “fixation” refers to specimen preservation and/or sterilization where cellular nucleic acid (DNA and RNA) integrity and cellular morphology are substantially maintained. Fixation can be performed either chemically using one or more solutions containing one or more fixing agent(s) and/or mechanically, such as for example by preparation of a smear on a microscope slide and subsequently heating the smear either by passing the slide through a flame or placing the slide on a heat block.

As used herein, “fixative agent or agents” refers a reagent, two or more reagents, a mixture of reagents, a formulation or even a process (with or without associated use of reagent(s) (including mixture(s) or formulation(s)) to treat microorganisms or cells to thereby preserve and/or prepare said microorganisms or cells for microscopic analysis. Some examples of fixative agents include paraformaldehyde, gluteraldehyde, methanol and ethanol. When more than one reagent is used to fix bacteria, the reagents can be added sequentially, simultaneously, or a combination of some reagents being added sequentially and some being added simultaneously. In some embodiments, methods disclosed herein can be practiced by contacting the sample with a fixative agent or reagents.

As used herein, “nucleic acid” refers to a polynucleobase strand formed from nucleotide subunits composed of a nucleobase, a ribose or 2′-deoxyribose sugar and a phosphate group. Some examples of nucleic acid are DNA and RNA.

As used herein “nucleic acid analog” refers to a polynucleobase strand formed from subunits wherein the subunits comprise a nucleobase and a sugar moiety that is not ribose or 2′-deoxyribose and/or a linkage (between the sugar units) that is not a phosphate group. A non-limiting example of a nucleic acid analog is a locked nucleic acid (LNA: See for example, U.S. Pat. Nos. 6,043,060, 7,053,199, 7,217,805 and 7,427,672). See: Janson and During, Peptide Nucleic Acids, Morpholinos and Related Antisense Biomolecules, Chapter 7, “Chemistry of Locked Nucleic Acids (LNA),” Springer Science & Business, 2006 for a summary of the chemistry of LNA.

As used herein the phrase “nucleic acid mimic” refers to a nucleobase containing polymer formed from subunits that comprise a nucleobase and a backbone structure that is not a sugar moiety (or that comprises a sugar moiety) but that can nevertheless sequence specifically bind to a nucleic acid. An example of a nucleic acid mimic is peptide nucleic acid (PNA: See for example, U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, WO92/20702 and WO92/20703). Another example of a nucleic acid mimic is a morpholino oligomer. (See Janson and During, Peptide Nucleic Acids, Morpholinos and Related Antisense Biomolecules, Chapter 6, “Morpholinos and PNAs Compared,” Springer Science & Business, 2006 for a discussion of the differences between PNAs and morpholinos.

It is to be understood that the scope of this invention is not limited to the use of “traditional” aminoethyl glycine-based PNA probes. The PNA probes include all possible PNA backbone configurations. As used herein, “peptide nucleic acid” or “PNA” refers to any oligomer or polymer comprising two or more PNA subunits (residues), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 6,201,103, 6,228,982 and 6,357,163; all of which are herein incorporated by reference. The term “peptide nucleic acid” or “PNA” can also apply to any oligomer or polymer segment comprising two or more subunits of those nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO 96/04000.

As used herein “nucleobase” refers to those naturally occurring and those non-naturally occurring heterocyclic moieties commonly used to generate polynucleobase strands that can sequence specifically bind to nucleic acids. Non-limiting examples of nucleobases include: adenine (“A”), cytosine (“C”), guanine (“G”), thymine (“T”), uracil (“U”), 5-propynyl-uracil, 2-thio-5-propynyl-uracil, 5-methylcytosine, pseudoisocytosine, 2-thiouracil, 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

As used herein “nucleobase sequence” refers to any nucleobase containing segment of a polynucleobase strand (e.g., a subsection of a polynucleobase strand). Non-limiting examples of suitable polynucleobase strands include oligodeoxynucleotides (e.g., DNA), oligoribonucleotides (e.g., RNA), peptide nucleic acids (PNA), PNA chimeras, nucleic acid analogs and/or nucleic acid mimics.

As used herein “nucleobase containing subunit” refers to a subunit of a polynucleobase strand that comprises a nucleobase. For oligonucleotides, the nucleobase containing subunit is a nucleotide. For other types of polynucleobase strands (e.g., nucleic acid analogs), the nucleobase containing subunit will be determined by the nature of the nucleobase containing subunits that make up said polynucleobase strand (i.e., a polynucleobase polymer).

As used herein “polynucleobase strand” refers to a complete single polymer strand comprising nucleobase containing subunits.

As used herein “probe” or “hybridization probe” refers to a composition that binds to a select target sequence. A “hybridization probe” is a probe that binds to its respective target sequence by hybridization. Non-limiting examples of probes include nucleic acid oligomers, (e.g., DNA, RNA, etc.) nucleic acid analog oligomers (e.g., locked nucleic acid (LNA)), nucleic acid mimic oligomers (e.g., peptide nucleic acid (PNA)), chimeras, and aptamers.

As used herein, “sequence specifically” refers to hybridization by base-pairing through hydrogen bonding. Non-limiting examples of standard base pairing include adenine base pairing with thymine or uracil and guanine base pairing with cytosine. Other non-limiting examples of base-pairing motifs include, but are not limited to: adenine base pairing with any of: 5-propynyl-uracil, 2-thio-5-propynyl-uracil, 2-thiouracil or 2-thiothymine; guanine base pairing with any of: 5-methylcytosine or pseudoisocytosine; cytosine base pairing with any of: hypoxanthine, N9-(7-deaza-guanine) or N9-(7-deaza-8-aza-guanine); thymine or uracil base pairing with any of: 2-aminopurine, N9-(2-amino-6-chloropurine) or N9-(2,6-diaminopurine); and N8-(7-deaza-8-aza-adenine), being a universal base, base-pairing with any other nucleobase, such as for example any of: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine) or N9-(7-deaza-8-aza-guanine) (See: Seela et al., Nucl. Acids, Res., 28(17): 3224-3232 (2000)). It is to be understood however that a probe or primer can hybridize with sequence specificity even in the presence of one or more point mutations, insertions or deletions such that the remaining complementary nucleobases are able to base-pair.

As used herein the term “subject” and “individual” are used interchangeably and include humans and animals (such as other mammalian subjects) that receive either prophylactic or therapeutic treatment. The term “subject” may, for example, refer to a human, to whom treatment is provided. A “non-human” subject may include mammals such as rats, mice, rabbits, sheep, cats, dogs, cows, pigs, and non-human primates.

As used herein “target” or “target sequence” refers to a nucleobase sequence (often a subsequence of the entire molecule) of a polynucleobase strand sought to be determined. The target sequence can, for example, be associated with a trait sought to be determined. The target sequence can, for example, associated with a species, genus, class, order, family, phylum or other classification of a microorganism or cell sought to be determined. Non-limiting examples of target sequences include mRNA, rRNA, plasmid DNA, viral nucleic acid and chromosomal DNA.

As used herein “trait” refers to any characteristic or property of a microorganism (e.g., bacteria) that can be determined by analysis of a target sequence that can be found within said microorganism. An example of one such trait is methicillin-resistance. Said trait is dependent on the presence of the mecA gene (i.e., the chromosomal DNA) and expression of said gene (e.g., by production of mRNA from said gene).

As used herein with respect to treatment of a disease or disorder, the term “treat,” “treatment” and “treating” are used interchangeably, and refer to preventing the development of the disease, or altering the course of the disease (for example, but not limited to, slowing the progression of the disease), or reversing a symptom of the disease or reducing one or more symptoms and/or one or more biochemical markers in a subject, preventing one or more symptoms from worsening or progressing, promoting recovery or improving prognosis, and/or preventing disease in a subject who is at risk thereof as well as slowing or reducing progression of existing disease.

### GENERAL

It is to be understood that the discussion set forth below in this “General” section can pertain to some, or to all, of the various embodiments of the invention described herein.

It should be understood that the order of steps or order for performing certain actions is immaterial so long as the present teachings remain operable or unless otherwise specified. Moreover, in some embodiments, two or more steps or actions can be conducted simultaneously so long as the present teachings remain operable or unless otherwise specified.

### Synthesis, Modification and Labeling of Nucleic Acids and Nucleic Acid Analogs

Nucleic acid oligomer (oligonucleotide and oligoribonucleotide) synthesis has become routine. For a detailed description of nucleic acid synthesis please see Gait, M. J., Oligonucleotide Synthesis: A Practical Approach, IRL Press, Oxford England (1984). Persons of ordinary skill in the art will recognize that labeled and unlabeled oligonucleotides (DNA, RNA and synthetic analogues thereof) are readily available. They can be synthesized using commercially available instrumentation and reagents or they can be purchased from commercial vendors of custom manufactured oligonucleotides.

### PNA Synthesis and Labeling

Methods for the chemical assembly of PNAs are well-known (See: U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053 and 6,107,470; all of which are herein incorporated by reference for their information pertaining to peptide nucleic acid synthesis, modification and labeling. Some non-limiting methods for labeling PNAs are described in U.S. Pat. No. 6,110,676, WO 99/22018, WO 99/21881, WO 99/49293 and WO 99/37670 are otherwise well known in the art of PNA synthesis. Chemicals and instrumentation for the support bound automated chemical assembly of peptide nucleic acids are commercially available. Likewise, labeled and unlabeled PNA oligomers are available from commercial vendors of custom PNA oligomers (See: See the worldwide web at: panagene.com/pna-oligomers.php, See the worldwide web at: biosyn.com/pna_custom.aspx or See the worldwide web at: crbdiscovery.com/pna/). Additional information on PNA synthesis and labeling can be found in Peter E. Nielsen, Peptide Nucleic Acids, Taylor and Francis, (2004).

Because a PNA is a polyamide, it has a C-terminus (carboxyl terminus) and an N-terminus (amino terminus). PNAs can be labeled at the C-terminus, the N-terminus or both the C-terminus and the N-terminus. For the purposes of the design of a hybridization probe suitable for antiparallel binding to a target (the preferred orientation), the N-terminus of the PNA oligomer is the equivalent of the 5′-hydroxyl terminus of an equivalent DNA or RNA oligonucleotide.

### Chimera Synthesis and Labeling/Modification

Chimeras are oligomers comprising subunits of different monomer types. In general, it is possible to use labeling techniques (with or without adaptation) applicable to the monomer types used to construct the chimera. Various labeled and unlabeled chimeric molecules are reported in the scientific literature or available from commercial sources (See: U.S. Pat. No. 6,316,230, See the worldwide web at: biosyn.com/PNA_{—} Synthesis.aspx, WO 2001/027326 and See the worldwide web at: sigmaaldrich.com/life-science/custom-oligos/dna-probes/product-lines/lna-probes.html). Therefore, persons of skill in the art can either prepare labeled chimeric molecules or purchase them from readily available sources.

### Labels/Modifications:

In general, any type of modification that that can be made to a synthetic oligomer can be used in the practice of the methods disclosed herein so long as they don't interfere with the hybridization or mass analysis steps. In some embodiments, the labels will be useful in the mass analysis and identifications relying thereon. In some embodiments, the labels can be used to affect the assay. It is to be understood that these need not be mutually exclusive outcomes such that the label or modification could be useful both in: 1) mass analysis and identifications relying thereon; and 2) affect the assay. It is also to be understood that generally there is no requirement that the hybridization probes comprise a label because they are being determined by their unique mass. However, in some embodiments, the nature of the label, if used, can be further confirmatory that the mass determined does indeed correspond to the hybridization probe and not a coincident background material.

The labels could also be selected to allow the hybridization probe to preferentially dissolve in a select solvent (e.g., organic solvents such as methanol or water or lipids such as mineral oil). In short, since the probe is being added to the sample it may be preferentially labeled so that it is easy to extract at the end of processing. For example, the probe could be made methanol soluble so that it could be extracted from the sample without also dissolving the cells or other cellular materials.

(i) Dyes

Any colorimetric, fluorescent or radioactive dye can be used to complement the practice methods disclosed herein even if they are not critical to the outcome of the assay. For example, as shown in Example 15, it is possible to use fluorescence to confirm that the bacteria are labeled with the hybridization probes prior to the mass analysis. In this way, use of the fluorescently labeled probes is complementary (and confirmatory) to the practice of the assay method—but not essential to its practice.

Non-limiting examples of fluorochromes (fluorophores) include 5(6)-carboxyfluorescein (Flu), 2′,4′,1,4,-tetrachlorofluorescein; and 2′,4′,5′,7′,1,4-hexachlorofluorescein, other fluorescein dyes (See: U.S. Pat. Nos. 5,188,934; 6,008,379; 6,020,481, incorporated herein by reference), 6-((7-amino-4-methylcoumarin-3-acetyl)amino)hexanoic acid (Cou), 5(and 6)-carboxy-X-rhodamine (Rox), other rhodamine dyes (See: U.S. Pat. Nos. 5,366,860; 5,847,162; 5,936,087; 6,051,719; 6,191,278; 6,248,884, incorporated herein by reference), benzophenoxazines (See: U.S. Pat. No. 6,140,500, incorporated herein by reference) Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.), other cyanine dyes (Kubista, WO 97/45539), 6-carboxy-4′,5′-dichloro-2′,7′-dimethoxyfluorescein (JOE), 5(6)-carboxy-tetramethyl rhodamine (Tamara), Dye 1 (FIG. 7), Dye2 (FIG. 7) or the Alexa dye series (Molecular Probes, Eugene, Oreg.).

(ii) Ligands & Anti-Ligands and Their Use in Hybridization Probe Capture

In some embodiments, the labels can be haptens and/or their corresponding binding partner. These can be generally referred to as ligand-anti-ligand interactions. Some non-limiting examples of haptens include 5(6)-carboxyfluorescein, 2,4-dinitrophenyl, digoxigenin, and biotin. In these cases the anti-ligand (or binding partner) can be an antibody raised to 5(6)-carboxyfluorescein, 2,4-dinitrophenyl and digoxigenin, respectively. However, for biotin there are natural and modified versions of streptavidin that are its suitable binding partner.

When used to label hybridization probes, these ligand-anti-ligand interactions can be used, for example, to capture hybridization probes.

In some embodiments, the capture can be used to remove excess hybridization probes in a manner to complements the washing step. By this we mean that the capture of unhybridized hybridization probes sequesters them (often on a solid support) to thereby remove them. In this way, the capture of unhybridized probes acts to substitute for, or work in harmony with, the washing step because it removes the unhybridized probes from the sample. An example of using the ligand-anti-ligand interactions to remove excess hybridization probe after the hybridization step has been performed can be found in Example 14, below.

When it is desirable to recover probes from intact cells for analysis by MS, probes comprising the ligand can be prepared as discussed above. Thus, in some embodiments, capture can be used to collect/recover hybridization probes post hybridization step, wherein the collected/recovered probes are the hybridization probes that bind to their respective targets sequences in the cells or microorganisms. Such methods are described in more detail below under the heading: “Methods Involving Recovery of Hybridization Probes.”

(iii) Positive or Negatively Charged Groups

A mass spectrometer differentiates between analytes based on their mass to charge (m/z) ratio. Thus, all analytes need to be charged in order to be detected in a mass spectrometer. In a mass spectrometer, even otherwise neutral analytes can (at least in a low abundance) become ionized both positively and negatively. A mass spectrometer can run in both positive and negative ion mode. That is, it can either analyze the sample for positive ions or it can analyze the sample for negative ions. Although neutral compounds become ionized within a mass spectrometer at least in low abundance, it is possible to introduce formal charges onto the analytes and thereby improve/increase their detectability since all of the analytes are charged.

Accordingly, in some embodiments, the hybridization probe or probes can incorporate a label that possesses a single formal positive or formal negative charged group. There is no advantage to introducing more than one charge as the mass spectrometer detects based on the mass to charge ratio such that every additional charge will only cause the observed mass to be proportionally lower based on the number of charges introduced. Nevertheless, the formal charge will typically improve the detectability of the hybridization probe over other possible contaminants of the sample that might have a corresponding or closely related mass to charge ratio because the formal charge will likely greatly improve signal strength for the ion associated with the hybridization probe as compared with the sample contaminates.

Alternatively, the hybridization probe or probes can incorporate a label that, although not formally charged, can be very easily ionized. Because it is easily ionized, there will be a greater prevalence of these ions in the mass spectrometer and this will improve the detectability of the hybridization probe over other possible contaminants of the sample that might have a corresponding or closely related mass to charge ratio because there will be improved signal strength for the ion associated with the hybridization probe as compared with the sample contaminants.

The formally charged or easily ionized labels can be introduced into PNA, for example, by incorporating an amino acid that is easily charged (e.g., lysine) or that comprises a formal charge (e.g., arginine). Other types of labels that can be used on PNAs or other probe types will be apparent to those of skill in the art. Moreover, oligomers comprising such labels are generally available for purchase from commercial vendors that engage in custom oligomer synthesis.

(iv) Signature Mass Tag

In some embodiments, the hybridization probes can comprise a signature mass tag. By ‘signature mass tag’ we mean a label that inherently possesses a unique mass signature that can be used in combination with the identified mass of the analyte (i.e., hybridization probe) to confirm that the observed peak indeed corresponds to the hybridization probe because contaminants peaks will lack the signature of the mass tag. A non-limiting example of a mass signature label are the iTRAQ reagents available from AB Sciex (can be found on the world wide web at: sciex.com/products/standards-and-reagents/itraq-reagents.xml?country=United%20States). These labeling reagents are designed in either a 4-plex or 8-plex configuration wherein each of the individual 4-plex or 8-plex configurations are of the same mass but each label of the -plex comprises a unique isotopic configuration that can be distinguished by additional mass analysis (i.e., unique mass signature). In this way, a hybridization probe bearing the mass signature tag can be analyzed for its expected mass and for the unique mass signature. If both the expected hybridization probe mass and mass signature are present, the identification has a very high degree of confidence.

### Hybridization Probes

Embodiments of this invention use hybridization probes (for example but not limited to10 to 20-mers in length). A hybridization probe can be any probe that can sequence specifically hybridize to rRNA, mRNA, plasmid DNA, viral nucleic acid, and/or chromosomal DNA target sequences of intact microorganisms or cells. Suitable hybridization probe types include, but are not limited to, nucleic acids, nucleic acid analogs and nucleic acid mimics. Hybridization probes that comprise a neutral backbone (e.g., PNA and certain other nucleic acid mimics) have been found to work particularly well. The nucleobase sequence of each probe is selected to hybridize to a target sequence that if present in the microorganism or cell will correlate with a condition of interest (e.g., will identify the microorganism, cell or a trait sought to be determined). Generally, PNA probes have been found to very effectively be used. The probes are permitted to hybridize (the “hybridization step”) to the target sequence within the intact microorganisms or cells of a sample of interest using hybridization techniques known and routinely used in the art (for example techniques used in ISH and FISH analysis). Such ISH and FISH protocols are well known to the ordinary practitioner and well developed in the art and need not be described in any detail herein.

In some embodiments, the hybridization probes can be aptamers or other specific binding agents of a known mass that are capable of selectively/specifically binding to a particular target sequence within a microorganism or cell that may be present in a sample. For simplicity, the binding of the aptamer or other specific binding agent (also for simplicity these will be referred to herein as a “probe,” “probes,” “hybridization probe” or “hybridization probes” depending on whether the singular or plural is required) to its molecular target is likewise referred to herein as a “hybridization step.”

Different probe types can require different periods of time for hybridization to occur. Those of skill in the art will appreciate that the period of time sufficient for the hybridization probes to sequence specifically hybridize to their respective target sequences is largely dependent on the probe type, the concentration of the hybridization probes in the solution and the hybridization conditions (e.g., the salt concentration, pH and temperature are very important variables). For example, PNA probes (and other probes (e.g., nucleic acid mimics) comprising a neutral backbone) will often hybridize sufficiently in 5-30 minutes whereas nucleic acid probes and some nucleic acid analog probes may take from 30 minutes to 2 hours (or more) to sufficiently hybridize to their respective target sequences. Those of skill in the art will be able to adjust the hybridization time so that sufficient hybridization occurs such that hybridization probes hybridize to their respective target sequences in sequence specific manner without enough non-specific binding to degrade assay performance to an unacceptable level.

Probes are typically used under ‘suitable hybridization conditions’. The extent and stringency of hybridization is controlled by a number of factors well known to those of ordinary skill in the art. These factors include the concentration of chemical denaturants such as formamide, ionic strength, detergent concentration, pH, the presence or absence of chaotropic agents, temperature, the concentrations of the probe(s) and quencher(s) and the time duration of the hybridization reaction. Suitable hybridization conditions can be experimentally determined by examining the effect of each of these factors on the extent and stringency of the hybridization reaction until conditions providing the required extent and stringency are found. When properly applied, suitable hybridization conditions result in sequence specific hybridization of a probe to its complementary target. As used herein, ‘suitable hybridization conditions’ refers to performing a hybridization under conditions sufficient for hybridization probes to hybridize to their respective target sequences in sequence specific manner without enough non-specific binding to degrade assay performance to an unacceptable level.

### Samples

A sample comprising bacteria, other microorganism or cells can come from any source. The source of a sample is not intended to be a limitation associated with the practice of any method disclosed herein.

Samples can be environmental samples such as samples from soil or water. Samples can come from consumer staples such as food, beverages or cosmetics. Samples can come from crime scenes (e.g., for forensic analysis). Samples can come from war zones or from sites of a suspected terrorist attack (for example, for testing of pathogenic bacteria, including weaponized bacteria (e.g., B. anthracis)). Samples can come from clinical sources. Samples from clinical sources can come from any source such as a human, a plant, a fish or an animal. Some non-limiting examples of clinical samples (from clinical sources) include blood, blood products, platelet preparations, pulmonary secretions, pus, sputum, spinal fluid, amniotic fluid, stool, urine, nasal swabs, throat swabs and the like, or portions thereof. Samples (including clinical samples) can include bacterial cultures and subcultures derived from any of the foregoing. Samples can include samples prepared, or partially prepared, for a particular analysis. For example, the sample may be a specimen that has been fixed and/or stored for a period of time.

### Bioactive Agent

A “bioactive agent” is any composition or mixture of compositions that can interact with an organism or cell to promote or inhibit a physiological response in the form of a change in a metabolic or genetic process. Bioactive agents may be generated endogenously or may be introduced to the cells or microorganisms exogenously. Some non-limiting examples of bioactive agents include antibiotics, antifungals, transcription regulators, translation regulators, cell wall synthesis inhibitors, enzyme inhibitors, DNA synthesis inhibitors, cell cycle inhibitors, proton pump inhibitors or any combination of any two or more of the foregoing.

### Wash or Washing

A wash or washing step is any process applied to a system with the intent of decreasing the concentration of chemical or substance within the system. Wash steps are typically performed by addition of a wash reagent. Wash reagents may either be passive, where the decrease in the concentration of the chemical or substance is due only to dilution, or may be active where the decrease in concentration of the chemical or substance is promoted by a component of the wash reagent. Washing efficiency may be increased by increasing the time (soaking) or temperature of the wash step. Multiple wash steps may be used to increase the dilution effect. Multiple wash steps may be performed using the same wash reagent, or multiple wash reagents. A wash step may be performed by applying a wash reagent to a system, then removing the wash reagent, or may be performed by applying a wash reagent to a system and leaving the wash reagent in place. In some instances a wash reagent left in place may be described as a step-down reagent, designed to dilute a component of the system to produce a chemical or biological effect. A wash step may be performed in which the wash reagent promotes a chemical or biological process.

A wash reagent is usually a solution. For example, a wash reagent can be a solution comprising alcohol(s), detergent(s), chaotrope(s), solvent(s), water, surfactant(s), enzyme(s) and any combination of any two or more of the foregoing.

As exemplified in Example 15, below, a so called ‘smart wash’ a hybridization probe or probes can be prepared to include a ligand that interacts with a complementary anti-ligand. The affinity in a smart wash may be though any chemical interaction, including hydrogen bonding, cation-pi bonding, pi stacking, covalent bonding, ionic pairing, metallic bonding, Van der Waals' bonding, dipole-dipole interactions, polar interactions or any combination of any two or more of the foregoing.

### Fixing or Fixation

Advantageously, Applicants have found that it is not always necessary to fix the cells or microorganisms to practice embodiments of this invention. Fixation is defined above and is generally carried out by use of a fixative agent or agents. Fixation may be achieved by chemical or physical means, or a combination thereof. Non-limiting examples of chemical fixation processes which may occur in/on a wall or membrane include cross-linking, dissolution or deionization. These processes may be promoted by the action of an enzyme, a denaturant, a solvent or an alcohol. Non-limiting examples of physical fixation processes include application of energy including heat, light or electric charge.

Fixing or Fixation may or may not be a separate step in the process of processing a sample according to the present invention. For example, heat fixation may occur coincidentally with the hybridization step.

Some non-limiting examples of agents that can be used for fixation include aldehydes such as formaldehyde, paraformaldehyde or glutaraldehyde and alcohols such as methanol, ethanol or isopropanol. Various fixative agents are commercially available.

Fixation of samples may take place on a slide, or other surface, or in a suspension. Non-limiting examples of methods used for fixation on slides include heat fixation, such as heating to 55° C. for 20 minutes and flame fixation. Often, a chemical and a physical fixation process are performed at the same time or in sequence, for example use of alcohol to fix a sample onto a slide followed by heat fixation to improve permeability.

### Lysing

Advantageously, Applicants have found that it is not always necessary to lyse the cells or microorganisms prior to performing the mass analysis to practice embodiments of this invention. Lysing refers to disruption of cellular walls or membranes within biological samples to the point that the cell is no longer intact. Lysing differs from fixing in that when lysing the cellular components are no longer substantially contained within the cell or microorganism. In some cases lysing involves separation of a cell into various chemically defined components such as proteins, lipids, etc. Lysing may be performed through various chemical or physical mechanisms. Enzymatic lysis using an enzyme such as lysozyme is possible. Likewise, chemical lysis using a solvent or detergent is also possible. Mechanical lysis using a process to exert force upon a cell such as cavitation, ultrasonication or shearing forces is also possible.

In some embodiments, cells are lysed to recover the hybridization probes after the hybridization step and washing step has been performed. Once recovered, they can be analyzed by mass spectrometry. In some embodiments, the hybridization probes can be concentrated prior to MS analysis. In some embodiments, the hybridization probes can be concentrated on a surface or support using hybridization probes bearing a ligand of a ligand/anti-ligand binding pair.

### Mass Spectrometry

The current prior art methods for microbe determination via MS require the instrumentation to resolve the ribosomal proteins in a complex sample. This requirement often pushes the sensitivity and resolution power of existing technology to their limits, especially for certain sample types such as blood cultures. The present invention avoids these pitfalls because it focuses on identifying a specific probe or probes associated with a condition of interest whereby the mass spectrometer can be tuned to detect the hybridization probe and properties of the hybridization probe or probes can be tuned to optimize for their identification.

The hybridization probes and methodology disclosed herein fits well into the “sweet spot” of most any available MS platform. Similarly, the masses of the hybridization probes may be adjusted to place them into an available “mass window” which may be available for a particular sample-type. Mass windows are areas of a mass spectrum where there are few or no mass peaks present. This approach could enable samples that are currently undetectable due to the presence of substances which interfere with the ribosomal proteins that are needed for microbe determinations. Furthermore this approach allows for less sample manipulation, thereby simplifying and perhaps increasing the sensitivity of the MS method for detection of microorganisms or cells in certain sample types.

Mass spectrometry (MS) refers to any process which measures the mass to charge ratio (m/z) of an ionized sample through a charged field in a vacuum. Applicants have shown that matrix-assisted laser desorption/ionization time-of-flight (MALDI TOF) can be used with embodiments of this invention. Similarly a MALDI TOF-TOF may be used and may preferably be used if, for example, where the hybridization probes comprise a mass signature tag. It is also possible to perform the MS analysis using electrospray ionization matrix assisted laser desorption ionization (ESI-MALDI). Mass spectrometry refers not only to instrumentation used, but also applies to the data generation method and the process leading to mass identifications. Such processes include, inter alia, preparing the sample, spotting the sample (in the case of MALDI-TOF), ionizing and sending the sample through a vacuum to a detector, detecting the signal and correlating the signal to a standard curve and then assigning m/z values to detected peaks. In some cases, mass spectrometry also refers to analysis of detected signals using software.

The operating parameters of a mass spectrometer may also be adjusted and tuned to optimize the detection methods. For example, MALDI-TOF instruments allow the electric field in the vacuum to be adjusted and toggled between negative ion mode and positive ion mode. Many MALDI-TOF mass spectrometers allow adjustment of parameters such as the gain of the detector. Some other possible adjustments include laser power intensity, ion gating, the number of laser shots accumulated per profile, and the total number of laser shots acquired. All these can be adjusted to improve practice of the MS analysis step of the currently disclosed methods.

### Performing Assays

According to some embodiments of the method, a hybridization probe can be designed chemically so that independent of its nucleobase sequence the mass of the probe is unique, while at the same time the probe sequence can be designed so as to be specific for, for example, a rRNA target of a particular species or genus of microorganism sought to be determined. Therefore, the presence of a particular unique probe mass within the mass spectrum of a sample that has been hybridized with hybridization probe and washed to remove excess and unbound hybridization probe will be diagnostic for the presence of the organism within the sample. The increased sensitivity of the spectrometer for said hybridization probes (as opposed to the ribosomal proteins) should permit the direct analysis of complex samples, such as blood cultures, without the need to first isolate a pure colony.

In practice, since one does not, a priori, know the identity of the organism in a blood culture then one may contact the cells/microorganism in, for example, a blood culture sample with a mixture of probes wherein each probe possesses a unique mass corresponding to a unique sequence for a condition of interest that may exist within the blood culture (i.e., the probes of the probe mixture may be selected to determine, for example, what cells/microorganism(s) is/are in the blood sample and/or what traits do cells and/or microorganisms of the blood culture possess). Only the hybridization probe or probes corresponding to the organism(s) actually present in the blood culture will be observed in the mass spectrum since other “non-binding” probes will be removed in the wash step. In the case of a mixed blood culture where more than one species is present, if the probes of the probe mixture are judiciously selected to determine different species, then a corresponding number of mass peaks for the probes specific for each species can be observed in the mass spectrum analysis.

For example, current blood sample analysis typically involves approximately 10 different “organism identifications” (and by extension approximately ten probes or probe sets could be used to analyze the majority (70% to 90%) of species (i.e., conditions of interest)) that are commonly required to be analyzed from positive blood cultures. Consequently, a rather simple probe set could be created and used in a MS-based assay according to embodiments of this invention that would be capable of identifying the majority of conditions of interest commonly determined for positive blood cultures. It is to be understood that in some embodiments, it may be desirable that some “organism identifications” using hybridization probes be made to the species level, whereas other “organism identifications” be made to a class of phenotypically or therapeutically similar species such as the coagulase negative staphylococci. Current methodologies routinely used in the art (e.g., sequence alignments and other standard probe design tools) permit the design of hybridization probes that can be uniquely tailored for the determination of each particular condition of interest. Many useful probes sequences are already known and routinely used in the art. The hybridization probes can typically be custom synthesized by commercial vendors and then be mixed to prepare a probe mixture that can be used to simultaneously determine all possible conditions of interest in a single MS analysis.

In some embodiments, determination of resistance (a “trait”) can be performed using probe-based MS identification wherein the hybridization probes are selected to bind to specific genes or gene transcripts instead of, for example, rRNA. For example, it is known that MRSA, upon exposure to methicillin type drugs such as oxacillin, will increase the production of mecA mRNA and PBP2a protein within the microorganism as a consequence of the presence of the mecA gene. This can be visually observed by PNA FISH using a mixture of fluorescently labeled PNA probes which hybridize to different sequences within the mecA mRNA. To convert this FISH-based assay to an MS-based assay one needs only to, for example, adjust the mass of each PNA probe through attachment of chemical tags (e.g., N- or C-terminal amino acids which do not impact the hybridization) such that each probe then has the same molecular weight. Specifically, if the mecA mRNA is present within the cells a peak should be present in the mass spectrum that corresponds to the adjusted mass of the probes in the mixture. Thus many different probes can contribute to the unique mass peak in the mass spectrum that is diagnostic for a particular condition of interest.

It is to be understood that if there is a highly expressed gene producing many copies of mRNA associated with a condition of interest only one probe may be required to provide sufficient MS-sensitivity whereas if it is a very low expression level of target mRNA sequence associated with a particular condition of interest, many probes may be needed and the mass of the many probes can be mass-adjusted so that they all comprise the same mass. In this way the intensity of the many different probes are additive and produce a proportionally larger signal in the MS spectrum. One can also produce a multiplex mixture of probes whereby the mixture contains different sets of probes where each set of probes is specific for a particular condition of interest and each individual probe of a specific set is mass-adjusted to possess the same mass and wherein the probes for each different condition of interest possess a unique mass as compared with the probes for all other conditions of interest sought to be determined using the multiplex mixture.

It is to be understood that embodiments of this invention permit one to target both rRNA and mRNA in the same assay. For example, a probe of unique mass can be designed to specifically hybridize to rRNA that is characteristic for S. aureus and it can be combined with a probe set that specifically hybridizes to mRNA associated with the presence of the mecA gene (that can be used to identify the trait of methicillin resistance) wherein the probes of the probe set comprise a unique mass as compared with the probe that specifically hybridizes to the rRNA of S. aureus. Thus, when both masses are observed in the MS spectrum, the sample can be said to contain MRSA. In some embodiments, further multiplexing of the assay can be achieved by, for example, adding an additional rRNA-directed probe for coagulase negative staphylococci to (CNS), wherein said rRNA-directed probe for coagulase negative staphylococci comprised still another unique mass as compared with the mass of any other probes of the mixture. In that way, the MS analysis can be used to distinguish S. aureus, from CNS from, MRSA and from MR-CNS.

The relative area of the mass peaks may provide additional important information. For example a relatively large mecA probe peak relative to the S. aureus rRNA probe peak may indicate a highly expressing MRSA whereas a small mecA probe peak relative to the rRNA probe peak may indicate a weakly expressing MRSA. Such information could be used to better diagnose patient conditions as well as select the amounts and types of antibiotic treatments administered to patients.

Certain traits within microorganisms are encoded on extrachromosomal plasmids within a microorganisms. For example, the carbapenemase NDM-1 which confers resistance to certain carbapenem drugs is often found encoded on a plasmid with the bacterium Klebsiella pneumoniae. Often the plasmid is present in many copies and while it will be possible to detect the mRNA expressed from the NDM-1 gene in the plasmid, it may further be possible to directly detect the gene by hybridization of a NDM-1 specific probe set to the DNA sequence of the NDM-1 gene. The ability to directly detect the NDM-1 gene may obviate the need to induce the expression of the gene (for example, by exposure of the microorganism to a drug such as a carbapenem) for the purpose of detecting its mRNA expression product, thereby resulting in a simplified assay. Likewise, if the sensitivity of the MS analyzer is quite good and/or the probes are tagged or chemically modified so as to make them very detectable by the spectrometer, then one may directly detect the presence of a single copy chromosomally encoded gene by using the aforementioned probe set where each member of the set is adjusted to the same mass and thereby contributes to the observed mass peak in the mass spectrum.

When using embodiments of this invention, the MS instrument and its corresponding software and results database may not need to be as complex as currently in use because the mass spectra of the invention may be less complex due to the intensity of the probe peaks and relative absence of peaks corresponding to, ribosomal and other proteins as well as other cellular debris.

The currently employed method of microorganism detection using ribosomal proteins requires establishment and maintenance of a database of mass spectrographs to which any new data is compared (the “natural spectra+database analysis approach”). An algorithm is used to compare the sample mass trace to the database to derive the identification of the new sample. Not only does this approach require frequent maintenance of the database, it may also require a separate database for every sample type (blood, stool, etc.). Because the unique probe masses observed in the mass spectrum represent defined compounds that are present because they were added to the sample and are not natural compounds (i.e., proteins) they are not subject to mutation, natural variation, evolution or other change which could confound results and which require frequent updating of the databases in the currently practiced methods. Additionally, the detection of hybridization probes that are specifically added and then detected in the MS trace allows the same database to be used across different samples types since masses that correlate with materials present in a particular sample type are generally of no concern.

Another pitfall of the current “natural spectra+database analysis approach,” which has been documented in the literature, is the frequent inability to resolve multi-organism (i.e., multiple species) mixtures since the algorithms are often not able to resolve spectra comprised of mass peaks from multiple organisms. Furthermore, to improve the certainty of a result a probe of unique sequence may be doubly “tagged” such that a given unique hybridization probe sequence produces two peaks in the mass spectrum, in this way a result where both peaks are observed for the same probe will be of higher confidence. We envision using internal control probes to correct for various sample handling steps or to improve quantitation. For example, internal control probes may help to detect mismatch hybrids if they occur, such that a control probe giving a 1× signal compared to a specific hybridization probe on the same target providing a 0.5× signal may indicate a mismatched hybrid (e.g., point mutation or a heterogeneous genotype).

The concept of double or multiple labeling may be further applied to maximize the amount of information derived from a particular sample (e.g., blood culture). Multiplex probe mixtures may include several probes which universally detect various groupings of microorganisms. The various groupings may include probes that are specific for various phylogenetic or phenotypic classes. Groupings may include, but are not limited to, bacteria, fungi, gram-positive bacteria, gram-negative bacteria, Candida genus, Enterobacteriaceae, Acinetobacter genus, coagulase negative staphylococci, or non-E. faecalis enterococci. Other groupings by Genus, Family, Order, Class, Kingdom, Phylum or other phylogenetic distinction are within the scope of the present invention.

As stated above, one may employ a strategy of doubly detecting many organisms or classes of organisms to improve the certainty of results or for other purposes. For example, one could design a multiplex probe set that ensures that vast majority of microorganisms present in a sample are detected with at least one probe; for instance a universal bacterial probe. This could act as a positive control for the assay.

It is also within the scope of this invention to provide very specific probe sets not necessarily to detect specific organisms in a sample (e.g., stool) but to detect or estimate total bacterial load as a means to diagnosis of a condition of interest in a patient. An example of a suitable probe set might be one that is designed as a multiplex probe set that is capable of detecting several higher order classes of targets, for example, Enterobacteriaceae (family), Firmicutes (phylum), Bacilli (class) and Clostridia (class). Use of this probe set in the method embodiments of this invention could be used to get a snapshot of the total bacterial load and composition from a sample, such as for example, stool.

Another example would be the use of a universal bacterial probe to directly and rapidly measure the bacterial load in a blood product such as a platelet preparation just prior to administration of the platelets to the patient. Current blood culture and respiratory methods are slow meaning increasing the risk that patients receive bacterially contaminated platelets because bacteria have grown to harmful levels during the time between when the platelets were sampled and the test results are available. Thus, even though a blood culture or respiratory test may show no, or low, level contamination, the actual contamination load in the platelets may be quite high when the patient is infused with them.

Although the selection of certain probe sets may be sample dependent, it is also within the scope of this invention to use the same probe set across various sample types. Specific detection of a particular organism of interest, for instance S. aureus, could be performed using the same probe or probe mixture regardless of the sample type. Where probes are released from intact cells prior to analysis, the resulting MS trace is not likely to differ across sample types. So not only can the same kit be used across different sample types, but the same data analysis may be used as well. The same will apply, as exemplified below, in samples in which probes are not released prior to analysis.

Because the methods employed by this invention do not require the comparison of obtained spectra to the spectra of known organisms to make a determination, it is an advantage of this invention that the computing power and user interface requirements of the associated MS analysis will likely be greatly minimized as compared to the current methodologies. Likewise, because certain probe types (e.g., PNAs) inherently “fly” well in a MS, we expect that MS hardware requirements could possibly be relaxed, for example, in terms of the laser strength, power usage, vacuum tube length, cost, etc. Because MS hardware is typically very costly, we expect that these relaxed requirements may result in the ability to use less expensive and perhaps smaller instruments than those currently used. Where specific masses are expected from a sample, and the number of possible specific probe masses to be determine is limited to a small number (for example, 10 distinct possibilities) it is easy to conceive of an instrument that could automatically “call” the result with high confidence using very basic software.

It is to be understood that the MS analysis is not limited to utilizing MALDI mass spectrometers but it may be used with any type of mass spectrometer that is able to detect the hybridization probes from the samples. For example, instead of MALDI interface electrospray or other interface may be used. Furthermore instead of a TOF, the mass analyzer could be a quadrupole, ion-trap or other ion separation modality. Virtually any ion source or ionization technique capable of introducing a probe into the MS platform may be used and the ion-separation and detection modes may be any that can be, or are typically used to detect probes such as PNA, oligonucleotides, peptides, and their analogues.

It is to be understood that embodiments of the methods disclosed herein could be used for a variety of non-medical uses such as pharmaceutical production, manufacturing, waste water analysis, food analysis, agriculture, veterinary diagnostics and industrial hygiene.

Another advantage of the present invention is the ability to “kit” a discreet set of probes that could be easily validated for a specific determination (e.g., MRSA analysis). The current use of MS which asks the broad question “what is in the sample” may be difficult to validate, since all possible answers have to be checked. A simplified, kitted, use of the technology asks the question “is this (analyte) in the sample,” where the number of possible analytes may be as few as one. This type of question may be much easier answer to validate for regulatory purposes. Thus, it is an advantage that the method embodiments of this invention may prove to be superior with respect to clinical validation/regulatory approval.

### Diagnostic Determinations

Diagnostic determination refers to making a diagnosis based upon the output of a test or tests that provide information on the state, trait, type, phenotype, genotype, strain, species, genus, phylogenetic distinction or other condition of interest of a cell or microorganism in a sample. A diagnostic determination can be used to guide the treatment or treatment recommendations applied to a subject from whom a sample is collected and examined by the practice of the invention disclosed herein. A diagnostic determination can be made by a technician, clinician, nurse or medical doctor.

### Therapeutic Recommendations:

Therapeutic recommendation refers to use of diagnostic determinations to inform recommendations for the proper treatment of a subject. A therapeutic recommendation can be made by a clinician, nurse or medical doctor.

### DETAILED DESCRIPTION OF EMBODIMENTS

It should be understood that the order of steps or order for performing certain actions is immaterial so long as the present teachings remain operable or unless otherwise specified. Moreover, in some embodiments, two or more steps or actions can be conducted simultaneously so long as the present teachings remain operable or unless otherwise specified.

### Methods Not Requiring Isolation of Cell Lysis Material

In one embodiment, this invention pertains to a method comprising: 1) contacting microorganisms or cells of a sample with one or more hybridization probes for a period of time and under conditions sufficient for said hybridization probes to sequence specifically hybridize to their respective target sequences, if present, within said microorganisms or cells; 2) washing said microorganisms or cells to remove excess hybridization probes; and 3) analyzing said microorganisms or cells by mass spectrometry to identify one or more hybridization probes retained within said microorganisms or cells at a time after performing step 2.

It is to be understood that in some embodiments the microorganisms or cells can be lysed after performing step 2, but before performing the analysis by mass spectrometry.

In other embodiments, whole microorganisms or cells can be introduced directly into the mass spectrometer. When introduced as whole cells or microorganisms, it is probable that the cells or microorganisms are lysed by operation of the mass spectrometer.

In some embodiments, the microorganisms or cells are fixed prior to, or during, the performance of step 1 of the aforementioned method. In general, the fixing step can be used to permeabilize the cells or microorganisms to the hybridization probes. Depending on the nature of the hybridization probes and the cells or microorganisms, fixation may or may not be required.

The nucleobase sequence of each of the one or more hybridization probes is selected to hybridize to a target sequence associated with a condition of interest. Hence, when said one or more hybridization probes hybridizes to a target sequence associated with a condition of interest then said method may further comprise determining one or more conditions of interest associated with said microorganisms or cells where said one or more conditions of interest correlates with said one or more hybridization probes identified in step 3) of the method.

In some embodiments, two or more hybridization probes are used. For example, at least two different hybridization probes can be selected such that each of the two hybridization probes hybridizes to a different complementary target sequence wherein each complementary target sequence correlates with a different condition of interest. Hence, if two hybridization probes are used, two conditions of interest can be determined, if three hybridization probes are used, three conditions of interest can be determined, if four hybridization probes are used, four conditions of interest can be determined, if five hybridization probes are used, five conditions of interest can be determined, if six hybridization probes are used, six conditions of interest can be determined, if seven hybridization probes are used, seven conditions of interest can be determined, if eight hybridization probes are used, eight conditions of interest can be determined, and so on. For example, in Examples 10-14, a mixture of 8 different PNA probes are used wherein at least seven of the PNA probes are selected to identify seven different bacteria if present in the sample.

In some embodiments, two or more hybridization probes are used wherein the at least two different hybridization probes can be selected to determine the same condition of interest. More specifically, the at least two or more hybridization probes can be selected such that each of the two hybridization probes hybridizes to a different complementary target sequence wherein each complementary target sequence correlates with the same condition of interest. In this way, identification of each of two probes acts as a confirmation of the result for the other probe.

In some embodiments, three or more hybridization probes are used wherein at least two different hybridization probes can be selected to determine the same condition of interest and wherein at least two of the hybridization probes hybridize to a different complementary target sequence wherein each complementary target sequence correlates with a different condition of interest. For example, in Examples 10-13, a mixture of 8 different PNA probes are used wherein at least seven of the PNA probes are selected to identify seven different bacteria if present in the sample but one probe is a universal bacterial probe. In this case, the universal probe will hybridize to any bacteria in the sample and each of the seven different bacteria probes will hybridize to the selected bacteria if present in the sample. In this way, two probes (one of the seven different bacteria probes and the universal probe) are required to identify any of the seven bacteria of interest in the sample. If only one of the specific hybridization probes is identified but not the other generic (universal) probe, the result is deemed inconclusive. If only the other generic (universal) probe is identified, the result is deemed conclusive for a bacteria not target specifically by the probe set.

### Methods Involving Producing a Cell Lysate

In some embodiments, this invention pertain to a method comprising: 1) contacting microorganisms or cells of a sample with one or more hybridization probes capable of determining a condition of interest within said microorganisms or cells for a period of time sufficient for said hybridization probes to sequence specifically hybridize to their respective target sequences associated with said condition of interest within said microorganisms or cells; 2) washing said microorganisms or cells to remove excess hybridization probes; 3) lysing said microorganisms or cells to produce a cell lysate; and 4) analyzing said cell lysate by MS to identify one or more hybridization probes contained in said cell lysate.

The nucleobase sequence of each of the one or more hybridization probes is selected to hybridize to a target sequence associated with a condition of interest. Hence, when said one or more hybridization probes hybridizes to a target sequence associated with a condition of interest then said method can further comprises determining one or more conditions of interest associated with said microorganisms or cells where said one or more conditions of interest correlates with said one or more hybridization probes identified in step 4) of the method.

It is to be understood that this method differs from the prior method in that the microorganisms or cell are at least partially lysed (by physical or chemical means) and a cell lysate is produced prior to the mass spectrometry step. In this embodiment, the cell lysate may optionally be recovered or it may be performed as an integrated part of the process such that it is not directly isolated/recovered (such as may be found in a flow through system).

### Methods Involving Recovery of Hybridization Probes

In some embodiments, this invention pertains to a method comprising: 1) contacting microorganisms or cells of a sample with one or more hybridization probes for a period of time and under conditions sufficient for said hybridization probes to sequence specifically hybridize to their respective target sequences, if present, within said microorganisms or cells; 2) washing said microorganisms or cells to remove excess hybridization probes; 3) treating said microorganisms or cells with heat and/or other denaturing conditions for a period of time sufficient to thereby cause said probe/target complexes to denature and said denatured hybridization probes to diffuse outside of the intact cells/microorganisms; 4) recovering said denatured hybridization probes that have diffused outside of said intact cells/microorganisms; and 5) analyzing said recovered denatured hybridization probes by mass spectrometry to thereby identify said recovered denatured hybridization probes.

The nucleobase sequence of each of the one or more recovered denatured hybridization probes is selected to hybridize to a target sequence associated with a condition of interest. Hence, one or more conditions of interest associated with said microorganisms or cells can also be determined where said one or more conditions of interest correlates with said one or more recovered denatured hybridization probes identified in step 5) of the method.

It is to be understood that this method differs from either of the aforementioned methods in that the hybridization probes are recovered from the intact cells/microorganisms after the hybridization and washing steps have been performed. By “recovered” in this method we mean that the denatured hybridization probes are extracted from the cells/microorganisms without lysing said cells or microorganisms. However, it is to be understood that, depending on the mass spectrometer used, the recovered denatured hybridization probes may or may not be directly isolated prior to performing the analysis by mass spectrometry. That is, in some embodiments, the recovered denatured hybridization probes will flow directly into the mass spectrometer for analysis without being first isolated.

Those of skill in the art will appreciate that a period of time sufficient to cause a probe/target complex to denature and for the denatured probes to diffuse outside of the intact cells/microorganisms will be highly condition dependent. For example if only heat is used, the process will be slower than if heat is combined with chemical denaturants (e.g., formamide). Similarly, the process will be slower if only chemical denaturants are used at ambient temperature (as compared with elevated temperature—e.g., 35-80° C.). Generally, this ‘denaturing and diffusion step’ can be accomplished from between 10 minutes to 2 hours.

Probes released from the cells by heating can be subsequently captured/concentrated through recovery of liquid surrounding them during the treatment by heat and/or denaturing conditions. For example, this will be accomplished by pelleting the cells. The recovered supernatant can be analyzed directly or concentrated if necessary.

Alternatively, recovered probes present in the supernatant can be collected by binding to complementary sequences immobilized on beads or other surfaces such as slides. Such a capture/concentration step can facilitate their introduction into the mass spectrometer and allow further washing to remove cellular contents or other potentially interfering agents or concentration of the probes into a small volume. In some embodiments the probes can be collected from the beads. In some embodiments, the beads can be directly analyzed in the MS instrument. In the foregoing discussion, cells/microorganism were lysed and processed such that any hybridized probe can be liberated with the ribosomal proteins and both would be available for MS analysis. For typical MS microbial sample preparation, the cells and their contents can be dissolved using a strong (volatile) acid solution such as 70% formic acid in combination with a solvent such as acetonitrile.

Rather than lysing the cells/microorganisms and liberating their contents, in some embodiments it may be advantageous to isolate the hybridization probes from the intact cells/microorganisms following the hybridization step and the washing step. By isolating the hybridization probes from the intact cells, it may be possible to further increase the sensitivity of the assay and/or to further multiplex the assay. This is because the isolation of the hybridization probes from the intact cells/microorganisms will eliminate much of the cellular debris (e.g., ribosomal and other proteins) that would otherwise generate background in the MS spectrum. A cleaner MS spectrum should permit increased sensitivity of the assay.

For example, once the cells/microorganisms have undergone the hybridization step and the wash step, any bound probes can be removed from the intact cells/microorganisms by, for example, heat treatment that denatures the hybridization probes from their respective target(s). In some embodiments, solvents, detergents, RNAses or combinations thereof (with or without added heat) can also be used to cause dissociation and dissolution of the hybridization probes without causing lysis of the cells/microorganisms or elution of a majority of the cellular contents. Because the hybridization probes are relatively small in size, they will easily pass though the cell membrane and into the solution once dissociated from their respective target sequences. Consequently, the resulting recovered probe solution can then be analyzed by MS. Because the resulting recovered probe solution is less complex, the sensitivity of the MS detector will be increased, as will the mass resolution. This means that assays where the hybridization probes are recovered from the intact cells/microorganisms should exhibit superior sensitivity and resolution as compared to embodiments where cells/microorganisms are lysed. Consequently it is expected that such assays will provide more diagnostic information than if the ribosomal proteins and other cellular materials are present.

### Methods Involving Only Identifying Sequestered Probes & Related Conditions of Interest

In a general sense, Applicants have surprisingly found that hybridization probes sequestered within cells and microorganisms can be detected by mass spectrometry in the presence of all the background/noise related to the cellular debris. That is, it has been surprisingly observed that cells and microorganisms comprising sequestered hybridization probes bound to their target sequences within intact cells can be introduced to the mass spectrometer as intact cells/microorganisms and the hybridization probes can be rapidly and conclusively identified by mass analysis despite all the presence of all the possibly contaminating cellular debris. As noted above, the nucleobase sequence of each of the one or more hybridization probes can be selected to hybridize to a target sequence associated with a condition of interest. Hence, when said one or more hybridization probes is identified, so is the condition of interest.

Hence, in some embodiments, this invention pertains to a method comprising: 1) identifying one or more hybridization probes sequestered within cells or microorganisms by performing mass spectrometry on said cells or microorganisms; and 2) determining one or more conditions of interest associated with said cells or microorganisms based on the so identified one or more hybridization probes.

### Conditions of Interest

In each of the aforementioned methods, one or more conditions of interest can be determined. In some embodiments, the condition of interest is a trait associated with the microorganism of cell. In some embodiments, the condition of interest is the determination of a species, genus, class, order, family, phylum or other classification of said microorganism or cell. An example of a trait is methicillin resistance. An example of a species of a microorganism is S. aureus. In some embodiments, hybridization probes are used to determine a trait as well as a species, genus, class, order, family, phylum or other classification of said microorganism or cell. Thus, for example, it is possible to identify methicillin resistant S. aureus in a sample by judicial selection of appropriate hybridization probes.

Methicillin resistant S. aureus is of significant clinical interest and its rapid accurate identification in a patient sample can improve patient outcomes. Hence, from this example it becomes clear that any of the aforementioned methods can further comprise making a diagnostic determination based upon the conditions of interest so determined. For example, the diagnostic determination can be that the patient from whom the sample was obtained can be said to have a methicillin resistant S. aureus infection.

Given that diagnostic determination, it may further be possible to make a treatment recommendation for said patient. In this case, a recommendation for treating the patient (a subject) with an effective amount of an antibiotic known to effective towards said methicillin resistant S. aureus would seem appropriate.

Hence, generally speaking any of the foregoing methods can further comprise making a treatment recommendation for a subject from whom the sample was obtained based upon said diagnostic determination. For example, the recommendation could be to treat the patient with an effective amount of an antibiotic known to effective towards methicillin resistant S. aureus.

In practice of any of the aforementioned methods, the hybridization probe or probes can be PNA probes or PNA chimera probes. In practice of any of the aforementioned methods, the hybridization probe or probes can be nucleic acid mimic probes, including nucleic acid mimics comprising a neutral backbone.

In practice of any of the aforementioned methods, the hybridization probe or probes can comprise a formal positively charged label or a formal negatively charged label. In practice of any of the aforementioned methods, the hybridization probe or probes can comprise a mass signature tag.

### ADVANTAGES

It is an advantage that embodiments of this invention permit MS to be used to perform accurate identification of microorganisms without the need to first isolate a pure colony or broth culture.

It is an advantage that embodiments of this invention permit MS to be used to perform accurate determination of antimicrobial resistance, susceptibility, toxigenicity or other attributes of microorganisms that cannot presently be determined by MS.

### EXAMPLES

Aspects of the present teachings can be further understood in light of the following examples, which should not be construed as limiting the scope of the present teachings in any way.

### 1. PROPHETIC EXAMPLES

### Example 1

### Preparation of Microorganisms from a Pure Isolate

Colonies are prepared on an agar plate containing media sufficient to support growth of microorganisms of interest. After a sufficient growth period at a sufficient growth temperature, one to three colonies of microorganism are harvested and suspended in 0.3 milliliters of deionized water. Nine hundred microliters of 100% ethanol are added; the mixture is mixed by inversion, and then centrifuged at 12,000×g for 3 minutes. The supernatant is decanted, the sample is centrifuged a second time, any remaining supernatant is carefully removed and the pellet is air dried.

### Example 2

### Preparation of Microorganisms from a Blood Culture

One milliliter of a positive blood culture is added to 0.2 milliliters of a 5% saponin solution, then votexed thoroughly to mix. After 5 minutes of incubation at room temperature, the tube is centrifuged at 16,600×g for 1 minute. The supernatant is decanted. The pellet is washed with 1 milliliter of deionized water, and re-centrifuged at 16,000×g for 1 minute. The supernatant is decanted, and the pellet is air dried.

### Example 3

### Viability Test of Prepared Microorganisms

The pellet produced from either Example 1 or Example 2 is resuspended in 0.1 milliliter of deionized water. 10 microliter of the suspension is used to inoculate either an agar plate or a liquid culture containing media sufficient to support growth of microorganisms. After a sufficient growth period at a sufficient growth temperature, either colonies are produced on the agar plate or the liquid culture has become turbid.

### Example 4

### Hybridization of PNA in Solution

The pellet produced from either Example 1 or Example 2 is resuspended in 20 microliter of deionized water. To the mixture is added 0.2 milliliter of PNA reagent (0.025 M Tris-HCl; 0.1 M NaCl; 50% (v/v) Methanol; 0.1% Sodium Dodecyl; 0.5% Yeast Extract Solution; 25-250 nM and one or more PNA probes, the nucleobase sequence of which is selected to determine a condition of interest). The contents are mixed by vortexing and the samples are incubated at 55° C. for 30 minutes. After 5 minute centrifugation at 10,000×g, the supernatant is removed and the pellet is resuspended in 0.5 milliliter of Wash Buffer (0.005 M Tris-HCl pH 9.0, 0.025 M NaCl and 0.1% Triton X-100). Samples are incubated at 55° C. for 10 minutes, re-pelleted, and re-suspended in 0.5 milliliter of Wash Buffer and heated at 55° C. for 10 minutes.

### Example 5

### Hybridization of PNA on a Solid Support

The pellet produced from either Example 1 or Example 2 is resuspended in 0.1 milliliter of deionized water. 10 microliter of sample and 1 drop of AdvanDx PNA FISH Fixation Solution (AdvanDx product No: CP0021) are mixed in a well on the surface of the solid support. The sample is fixed by placing it at 55° C. for 20 min, then in 96% (v/v) ethanol for 5 minutes, then air dried. Hybridization is performed by adding 1 drop of a PNA FISH hybridization solution (such as S. aureus PNA FISH, KT001, AdvanDx Woburn, Mass.) and a cover slip, then incubating at 55° C. for 30 minutes. The coverslip is removed, and the sample is washed for 30 minutes at 55° C. in 1× PNA FISH Wash Solution. Optionally, the wash step is repeated, and the sample is air dried.

### Example 6

### Detection of Bound PNA by Mass Spectrometry

The method provides a means to detect PNA bound in a previous hybridization step to be detected by first dissolving the detected microorganisms in a solvent.

The solution produced from Example 4 is pelleted by 5 minute centrifugation at 10,000×g. Between five and fifty microliters of 70% formic acid is added to the pellet dependent on the pellet size, followed by an equal volume of acetonitrile. The sample is centrifuged again at 12,000 g for 3 minutes. 0.5 to 5.0 microliters of the supernatant are spotted on a solid surface and air dried. The sample is overlaid with matrix (saturated a cyano-4-hydroxycinnamic acid, 50% acetonitrile, 2.5% trifluoroacetic acid) and air dried. The sample is analyzed using a MALDI-TOF mass spectrometer and the PNA probes present in the sample are identified and used to determine a condition of interest associated therewith.

### Example 7

### Detection of Released PNA by Mass Spectrometry

The method provides a means to detect PNA bound in a previous hybridization step to be detected by releasing the PNA into a solvent.

The solution produced from Example 4 is pelleted by 5 minute centrifugation at 10,000×g. 10 to 100 microliters of 1M ammonia in methanol is added to the pellet, votexed, then incubated at 40° C. for 10 to 20 minutes to release the PNA from the microorganisms. The sample is centrifuged at 12,000×g for 3 minutes. 0.5 to 5.0 microliters of the supernatant is spotted on a solid surface and air dried. The sample is overlaid with matrix (saturated a cyano-4-hydroxycinnamic acid, 50% acetonitrile, 2.5% trifluoroacetic acid) and air dried. The sample is analyzed using a MALDI-TOF mass spectrometer and the PNA probes present in the sample are identified and used to determine a condition of interest associated therewith.

### Example 8

### Determination of Resistance by Detection of Bound PNA by Mass Spectrometry

An aliquot of a blood culture is combined with a solution containing an antibiotic of interest. The combined solutions are incubated to allow exposure of the organisms in the culture to the drug for a period and at a temperature sufficient to stimulate a physiological reaction to the drug. One milliliter of the blood culture mixture is added to 0.2 milliliters of a 5% saponin solution, then votexed thoroughly to mix. After 5 minutes incubation at room temperature, the tube is centrifuged at 16,600×g for 1 minute. The supernatant is decanted. The pellet is washed with 1 milliliter of deionized water, and re-centrifuged at 16,000×g for 1 minute. The supernatant is decanted, and the pellet is resuspended in 20 microliter of deionized water. To the mixture is added 0.2 milliliter of PNA reagent (0.025 M Tris-HCl; 0.1 M NaCl; 50% (v/v) Methanol; 0.1% Sodium Dodecyl Sulfate; 0.5% Yeast Extract Solution; 25-250 nM of one or more PNA probes). One or more of the PNA probes may be complementary to rRNA sequences (for identification of the organism). One or more of the PNA probes may be complementary to the mRNA of a resistance gene. If more than one probe is used for identification of the resistance gene it is preferred to design the probes such that some or all of them have the same mass. The contents are mixed by vortexing and the samples are incubated at 55° C. for 30 minutes. After 5 minute centrifugation at 10,000×g, the supernatant is removed and the pellet is resuspended in 0.5 milliliter of Wash Buffer (0.005 M Tris-HCl pH 9.0, 0.025 M NaCl and 0.1% Triton X-100). Samples are incubated at 55° C. for 10 minutes, re-pelleted, and re-suspended in 0.5 milliliter of Wash Buffer and heated at 55° C. for 10 minutes. The solution is pelleted by 5 minute centrifugation at 10,000×g. Between five and fifty microliters of 70% formic acid is added to the pellet dependent on the pellet size, followed by an equal volume of acetonitrile. The sample is centrifuged again at 12,000 g for 3 minutes. 0.5 to 5.0 microliters of the supernatant are spotted on a solid surface and air dried. The sample is overlaid with matrix (saturated a cyano-4-hydroxycinnamic acid, 50% acetonitrile, 2.5% trifluoroacetic acid) and air dried. The sample is analyzed using a MALDI-TOF mass spectrometer and the PNA probes present in the sample are identified and used to determine a condition(s) of interest associated therewith.

### Example 9

### Analyzing Microorganisms from Low-Titer Samples

In many cases, it is of interest to analyze microorganisms from samples in which they are low in number, such as blood or water. It is possible to concentrate the microorganisms by filtering such samples through a membrane filter having a pore size small enough to retain the bacteria of interest. For blood, such filtration requires that the blood cells first be lysed and the resulting cell debris treated to solubilize it. Selective lysis of the blood using saponin and high-frequency ultrasound accomplishes this requirement.

Lysis solution is prepared by adding 115 mg of saponin to 10 mL of 0.1M sodium phosphate buffer, pH 8 and vortexing to dissolve. 11.25 Units/mL of proteinase are added and vortexed briefly to dissolve. The solution is filtered using a 0.2 μm, 32 mm, PES syringe filter.

Blood samples are prepared by adding 1 mL of lysis solution and 1 mL of blood to a 3 mL, round bottom, glass Covaris tube. The samples are mixed by inversion.

The bath on a Covaris S2 Sonicator is filled with deionized water, heated to 37° C., and degassed for 30 minutes. The tubes are loaded into the custom tube holder designed to fix the X and Y axis. The samples are warmed and mixed for 100 seconds at an intensity of 1, 10% duty cycle, and 1000 cycles per burst. Then the intensity is increased to 2 for 60 seconds. Finally, the cycles per burst is decreased to 200 for 60 seconds.

The lysate is concentrated on a metal coated polycarbonate track etched membrane (PCTE) filter with a pore size of 0.6 microns. The metal can be gold or other suitable metal.

Concentration Method

Filter entire lysate using a vacuum equivalent 5 to 15 inches of Hg. Rinse filter and holder 3 times with 830 μL each of 1×PBS while vacuuming. Turn off and purge vacuum.

Optional Growth Step

Place the membrane filter onto an agar plate (composition chosen to be suitable for the microorganisms of interest). Incubate at 37° C. for 2-6 hours to allow growth of microcolonies.

Optional Hybridization and Wash Steps

Place the membrane filter into a thermostatted holder fitted with a disposable plastic tube that allows fluid to be dispensed onto the membrane. Filter PNA FISH Flow Hybridization Buffer immediately prior to use with a 13 mm, 0.2 μm, polytetrafluorethylene (PTFE) syringe filter. Add 400 μL of filtered or PNA FISH Flow Hybridization Buffer containing 100 nM to 500 nM or 50 nM probe for bacteria or yeast respectively to the holder. Cover the holder to prevent evaporation. Heat the retentate and hybridization buffer in the holder for 30 minutes at 55° C. Vacuum away hybridization buffer. Turn off and purge vacuum. Add 500 μL of PNA FISH Flow Wash Buffer to the holder. Cover holder to prevent evaporation. Heat the retentate and wash buffer in the holder for 10 minutes at 55° C. Vacuum away wash buffer. Turn off and purge vacuum. Optionally repeat steps.

Analysis

Dry the membrane filter with trapped (optionally hybridized and washed) microorganisms (optionally microcolonies). The membrane is overlaid with matrix (for example saturated a cyano-4-hydroxycinnamic acid, 50% acetonitrile, 2.5% trifluoroacetic acid) and air dried. The membrane is placed on the MALDI sample plate and held in place using a metal ring that establishes a conductive path from the metal coating on the membrane to the sample plate. The sample is analyzed using a MALDI-TOF mass spectrometer and the PNA probes present in the sample are identified and used to determine a condition(s) of interest associated therewith.

### 2. NON-PROPHETIC EXAMPLES

### Materials and Methods for Examples 10-14

Blood Lysis Solution: This reagent solution was prepared to contain 5% saponin, 10% sodium dodecyl sulfate (SDS), 91.575 mM Na₂HPO₄, and 6.8 mM NaH₂PO₄.

Hybridization Solution: This reagent solution was prepared to contain 505 mM Tris(hydroxymethyl)aminomethane (Tris), 1% tetradecyltrimethylammonium bromide (TTAB), 0.1% Tritin-X-100, 10 mM calcium chloride and 15 mM sodium chloride. Final pH was between 8.9 and 9.15. Subsequent probe solutions were made by addition of probes to a final concentration of 25 to 50 nM. In general, individual probes comprised approximately 0.1% of the final volume of the reagent.

Wash Solution: This reagent solution was prepared to contain 0.1% Triton-X-100, 200 mM sodium chloride, and 15 mM Tris pH 9.0. The solution was titrated with 36.5-38% hydrochloric acid to a final pH between 8.8 and 9.1.

Bind and Wash Buffer: This reagent solution was prepared to contain 5.0 mM Tris-HCl (pH 7.5), 0.5 mM EDTA and 1.0M NaCl.

Matrix Solution: This saturated reagent solution was prepared to contain 20-30 mg/mL α-Cyano-4-hydroxycinnamic acid, 2.5% TFA and 50% acetonitrile.

General Procedure for Detection of Microorganisms
1. Obtain 5 mL sample (primary or culture).
2. Pellet human cells in a swinging bucket centrifuge for 10 min at 150×g.
3. Using a pipette, carefully remove and retain the top-most 1 mL of sample.
4. Transfer sample to a new microcentrifuge tube, and add 200 μL Blood Lysis Solution, mix by inversion.
5. Incubate for 5 min at room temperature (˜20° C.).
6. Pellet microorganisms in a fixed angle centrifuge for 1 min at 15,000×g.
7. Remove and discard supernatant. Rinse pellet with 1 mL deionized water.
8. Pellet microorganisms in a fixed angle centrifuge for 1 min at 15,000×g.
9. Remove and discard supernatant. Add 500 μL Hybridization Solution, vortex to resuspend pellet, incubate in a water bath at 55° C. for 15 min.
10. Pellet microorganisms in a fixed angle centrifuge for 1 min at 15,000×g.
11. Remove and discard supernatant.
12. Resuspend pellet in 500 μL Wash Solution, incubate in a water bath at 55° C. for 10 min.
13. Pellet microorganisms in a fixed angle centrifuge for 1 min at 15,000×g.
14. Remove and discard supernatant.
15. Repeat wash step (steps 12-14).
16. Pellet microorganisms in a fixed angle centrifuge for 1 min at 15,000×g.
17. Resuspend pellet with 300 μL 0.1% trifluoroacetic acid (TFA).
18. Pellet microorganisms in a fixed angle centrifuge for 1 min at 15,000×g.
19. Remove and discard supernatant.
20. Resuspend pellet in 15 μL 0.1% TFA.
21. Spot 1 μL of cellular suspension onto the MALDI plate.
22. Overlay with 1 μL Matrix Solution (α-Cyano-4-hydroxycinnamic acid).

Alternative Procedure to Preforming Steps 12-15, above (the “Smart Wash Procedure”)
1. After step 11 of Detection of Microorganisms in a Blood Culture (above), resuspend the pellet in 50 μL of Bind & Wash Buffer.
2. Add 50 μL of streptavidin coated magnetic beads in Bind and Wash Buffer (M280 Dyna Beads, Invitrogen Cat #11205D) to the pellet, pipetting to mix.
3. Incubate for 5 min at room temperature (˜20° C.).
4. Position tube on magnet for 3 min.
5. Aspirate supernatant with a pipet, and deposit into a new microcentrifuge tube.
6. Resume Detection of Microorganisms in a Blood Culture (above), starting at step 16.

### PNA Probes

All hybridization probes used in Examples 10-15 were PNA probes. PNA probes were prepared from monomers though standard peptide synthesis methods. In most cases, PNAs were labeled on the amine terminus with an arginine moiety and on the carboxyl terminus with a biotin moiety attached through a lysine residue. In two cases fluorescein (Flu) labeled probes are described. Probes are designed to detect particular species of microorganisms or groups of microorganisms. The probe identifications, masses, their specific target and nucleobase sequences are described in Table 1.

TABLE 1

| SEQ | | | Labels & |
|---|---|---|---|
| ID | | | Nucleobase |
| # | Mass | Target | Sequence |
| 1 | 4095.1 | Non-aureus | Arg-AGACGTGCATAG |
| | | Staphylococci | T-Lys(Biotin) |
| 2 | 4313.3 | Enterococcus | Arg-CCTTCTGATGGG |
| | | faecium | CA-Lys(Biotin) |
| 3 | 4353.3 | Enterococcus | Arg-CCTCTGATGGGT |
| | | faecalis | AG-Lys(Biotin) |
| 4 | 4471.5 | Klebsiella | Arg-CACCTACACACC |
| | | pneumoniae | AGC-Lys(Biotin) |
| 5 | 4506.5 | Staphylococcus | Arg-GCTTCTCGTCCG |
| | | aureus | TTC-Lys(Biotin) |
| 6 | 4549.5 | Universal | Arg-CTGCCTCCCGTA |
| | | bacteria | GGA-Lys(Biotin) |
| 7 | 4606.6 | Pseudomonas | Arg-CTGAATCCAGGA |
| | | aeruginosa | GCA-Lys(Biotin) |
| 8 | 4645.6 | Escherichia | Arg-TCAATGAGCAAA |
| | | coli | GGT-Lys(Biotin) |
| 9 | 4354.1 | Staphylococcus | Flu-OO-GCTTCTCGT |
| | | aureus | CCGTTC |
| 10 | 5156.0 | Escherichia | Flu-TCAATGAGCAAA |
| | | coli | GGT-EE |

Key:
Arg = Arginine,
Lys = Lysine,
E = commercial solubility enhancer;
Flu = 5(6)-carboxyfluorescein

### MALDI-TOF Analysis

Samples were spotted onto a steel plate (sometimes referred to as a target) and overlaid with Matrix Solution. An external calibration standard, comprised of 100 to 500 fmol/μl each angiotensin II (human), P14R (synthetic peptide), ACTH fragment 18-39 (human), insulin oxidized B chain (bovine) and insulin (bovine), was also spotted on the plate and overlaid with Matrix Solution. The instrument was calibrated to this calibration standard regularly. To generate mass spectra, the plate was loaded into the MALDI-TOF instrument and placed under vacuum. A nitrogen laser was fired onto each sample. Molecules within the ablated sample traveled through the vacuum and were detected. Instrument manufacturer's software was used to convert minute changes in voltage recorded by the detector into a mass spectrum. Masses (measured as a mass to charge (or m/z ratio) were displayed on a digital interface.

Analysis of the mass spectra was arbitrarily limited in the examples herein to the range of approximately 4000 to 4700 m/z (mass/charge) which, for singly labeled molecules, translate to 4000 to 4700 daltons. This mass limitation was reasonably expected to simplify the analysis, since the masses of the hybridization probes of interest lie within that range. The method is by no means restricted to this range.

Individual mass peaks, sometimes associated with shoulders, are displayed graphically from low to high m/z signal. The software describes the height of the peaks in “% max” which is the percentage of each peak compared to the maximum peak, where the maximum peak, by definition, equals 100% of % max. Other options for displaying the data are available and are within the scope of this invention.

The accuracy of a mass spectrometer is dependent on the calibration of the instrument. Each time an instrument is calibrated, the accuracy is at a peak. Over time and space, the accuracy begins to fall off. As such, frequent calibration is required. In practice, calibration is performed against an external standard and for the present examples, accuracies of within 10 daltons or 0.2% m/z was acceptable.

### Example 10

### Detection of Various Species of Bacteria in Culture

Through efforts to implement the disclosed prophetic examples, improvements/enhancements were made in many aspects of the experimental method. Surprisingly, Applicants have found that it was possible to diagnostically detect probe signals by mass spectrometry without either the need for a separate fixation step, or removal of the sequestered hybridization probes from the microorganisms or cells prior to mass spectrometry analysis. For example, reagents were developed which allowed sufficient permeabilization of cells during the hybridization step to allow penetration of hybridization probes, obviating the requirement for a separate treatment with a fixative agent or agents. The improved methods leave cells or microorganisms essentially intact throughout the process of hybridizing probes and washing cells or microorganisms, a step used to remove excess (unhybridized/sequestered) hybridization probes. The improved methods were also optimized to effectively eliminate (or at least greatly minimize) detection of signals from cellular components of similar masses. As a result, intact cells were found to be successfully introduced directly to the mass spectrometer for analysis.

A Hybridization Solution containing eight PNA oligomers from Table 1 (Probes of SEQ ID NO: 1-8) was prepared with 50 nM each PNA probe, except probe B which was at 25 nM. Individual Tryptic Soy Broth (TSB) cultures of seven organisms including Staphylococcus epidermidis, Enterococcus faecalis, Enterococcus faecium, Klebsiella pneumoniae, Staphylococcus aureus, Pseudomonas aeruginosa, and Escherichia coli were prepared. One milliliter of each TSB culture was treated as described above under the heading: General Procedure for Detection of Microorganisms, except that the process was started at step 8. Spectra were scored for the presentation of peaks corresponding to the probe masses presented in Table 1, +/−10 daltons. Table 2 displays the two highest peaks observed for each sample.

TABLE 2

| | Peak | Peak | |
|---|---|---|---|
| Sample | 1 | 2 | Interpreted identification |
| Staphylococcus | 4100 | 4555 | Non-aureus Staphylococci |
| epidermidis | | | |
| Enterococcus faecium | 4318 | 4555 | Enterococcus faecium |
| Enterococcus faecalis | 4357 | 4554 | Enterococcus faecalis |
| Klebsiella pneumoniae | 4552 | 4474 | Klebsiella pneumoniae |
| Staphylococcus aureus | 4509 | 4551 | Staphylococcus aureus |
| Pseudomonas aeruginosa | 4554 | 4611 | Pseudomonas aeruginosa |
| Escherichia coli | 4551 | 4647 | Escherichia coli |

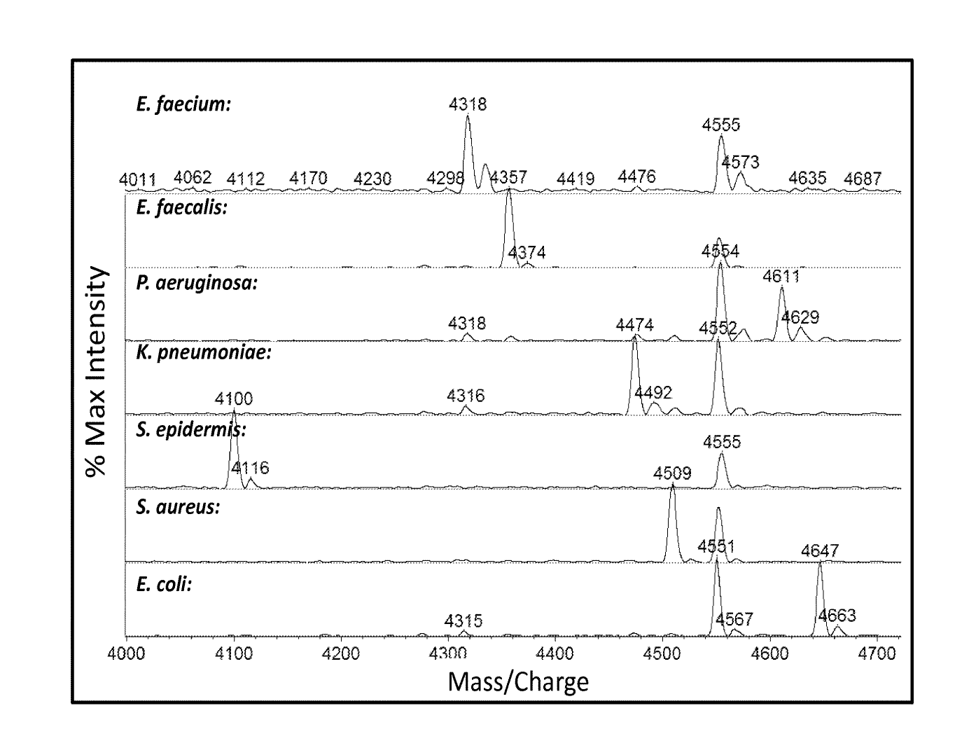

The data in Table 2 (also displayed in FIG. 1) demonstrate that the method was successfully applied to TSB cultures of microorganisms. Of the two major peaks (m/z) recorded for each sample, one corresponded to the species specific PNA probe and one corresponded to the universal bacterial probe, mass 4549 (+/−10 was within the range of the calibration errors). It is known from experimental results using the universal bacterial probe performed by the Applicants that the majority of bacteria detected in clinical samples are positively detected by the universal bacteria probe. Therefore, this probe was used as an internal experimental control. Any sample not showing a m/z peak correlating to the universal bacterial probe mass was considered negative (i.e., no bacteria detected). The mass displayed in Table 2 “Peak 1” gave the greatest signal for each pair. Some minor peaks were observed which may correspond to ribosomal proteins. In two samples, K. pneumoniae and P. aeruginosa, very small peaks were observed which correlate to the mass of the E. faecium probe, suggesting a weak cross reaction.

Note: As a result of oxidation of the biotin label, many of the probe peaks are associated with a shoulder peak which is approximately 16 daltons larger. These secondary peaks were ignored in the analysis.

These data demonstrate several features of the present invention. For example, multiple probes can be tested in parallel to detect multiple analytes. Although only a single “universal” probe was tested in this instance, those familiar with the art of molecular analysis would understand that several probes of different mass and specific for a particular organism or organisms could be applied at once to increase the cumulative specificity of a particular probe set. Also, the example demonstrates how a single probe mixture (cocktail) can be used to produce multiple identifications.

While the conventional methods are complicated, requiring a database and software to analyze the entirety of the spectral trace by comparison to standard spectra, the present method is remarkably simple and easily interpreted by man or machine.

### Example 11

### Detection of Various Species of Bacteria Directly from Blood Culture

The same Hybridization Solution as prepared according to Example 10 was applied to nine (9) blood culture samples of actual hospitalized patients obtained from a hospital microbiology lab. Routine identifications were unknown at the time of testing. Sample processing and hybridization was carried out as described under the heading: General Procedure for Detection of Microorganisms. The full method includes the use of Blood Lysis Solution to lyse a portion of the blood cells in the sample and a centrifugation step to effectively separate the microbial cells from the pelleted human blood cells and cell components. Identification was made based on the two most prominent peaks, and then compared to the clinical identifications obtained from the clinical lab. Peaks below approximately 10% maximum signal were ignored. Results are recorded in Table 3.

TABLE 3

| | Peak | Peak | | |
|---|---|---|---|---|
| Sample# | 1 | 2 | MS Interpretation | Clinical Identification |
| 1856 | 4354 | 4551 | Enterococcus faecalis | Enterococcus faecalis |
| 1858 | 4096 | 4550 | Non-aureus | Coagulase Negative |
| | | | Staphylococci | Staphylococcus |
| 1859 | 4550 | 4314 | Enterococcus faecium | Enterococcus faecium |
| 1860 | 4550 | 4314 | Enterococcus faecium | Enterococcus faecium |
| 1867 | 4550 | 4646 | Escherichia coli | Escherichia coli |
| 1868 | 4550 | 4646 | Escherichia coli | Escherichia coli |
| 1871 | 4508 | 4551 | Staphylococcus | Staphylococcus aureus |
| | | | aureus | |
| 1872 | 4509 | 4551 | Staphylococcus | Staphylococcus aureus |
| | | | aureus | |
| 1905 | 4552 | — | Bacteria | Micrococcus spp. |

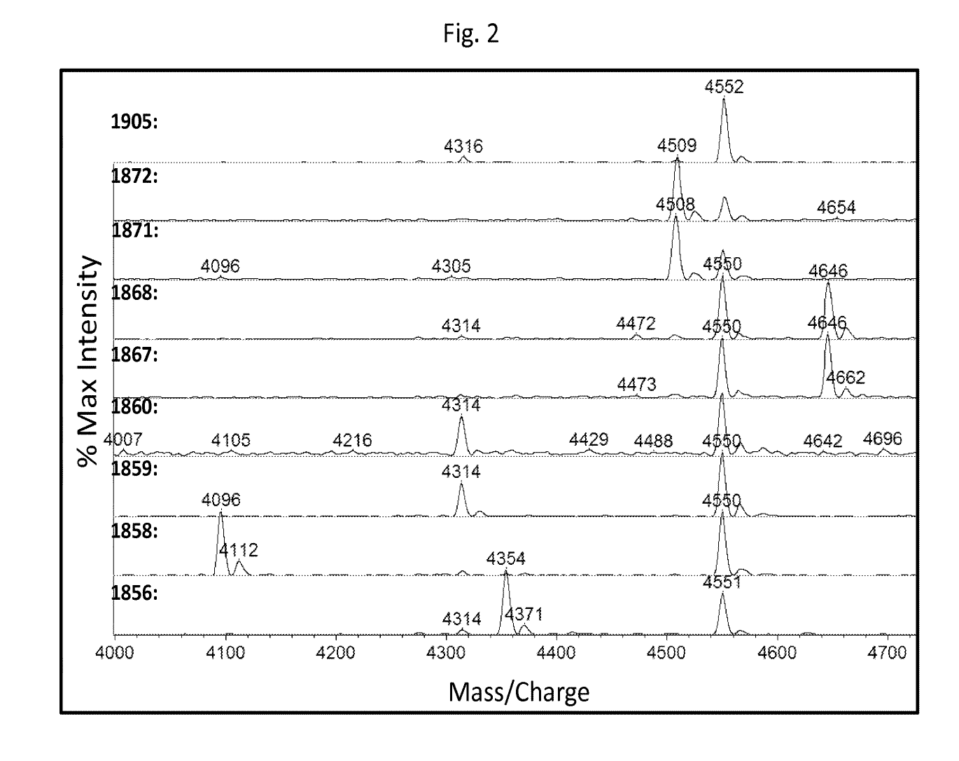

Data are presented in FIG. 2 and Table 3. All samples presented two prominent peaks, except one, which only displayed a strong peak for the universal bacteria probe. The mass displayed in Table 3 “Peak 1” gave the greatest signal for each pair. Interpretations were compared to the clinical identifications revealing that all samples were called correctly by the MS analysis. The sample which was only positive for the universal bacteria probe was identified as a Micrococcus spp. Close inspection revealed a weak peak corresponding to the E. faecium probe (less than 10% of the universal bacteria probe peak intensity), indicating a weak cross-reaction. These data clearly demonstrate that the methods developed for spiked strains in TSB culture can be successfully applied to actual clinical blood culture samples, merely by addition of steps to lyse blood cells in the sample and effectively separate the bacteria.

### Example 12

### Detection of Various Species of Bacteria Directly from Urine

The same Hybridization Solution as prepared according to Example 10 was applied to two (2) urine samples obtained from a hospital microbiology lab. Sample processing and hybridization was carried out as described under the heading: General Procedure for Detection of Microorganisms, except that steps 4 and 5 were eliminated. Identification calls were made based on the two most prominent peaks, and then compared to the clinical identifications obtained from the clinical lab. Results are recorded in Table 4. The mass displayed in Table 4 “Peak 1” gave the greatest signal for each pair.

TABLE 4

| | Peak | Peak | | |
|---|---|---|---|---|
| Sample# | 1 | 2 | MS Interpretation | Clinical Identification |
| U1 | 4552 | 4474 | Klebsiella pneumoniae | Klebsiella pneumoniae |
| U2 | 4356 | 4552 | Enterococcus faecalis | Enterococcus faecalis |

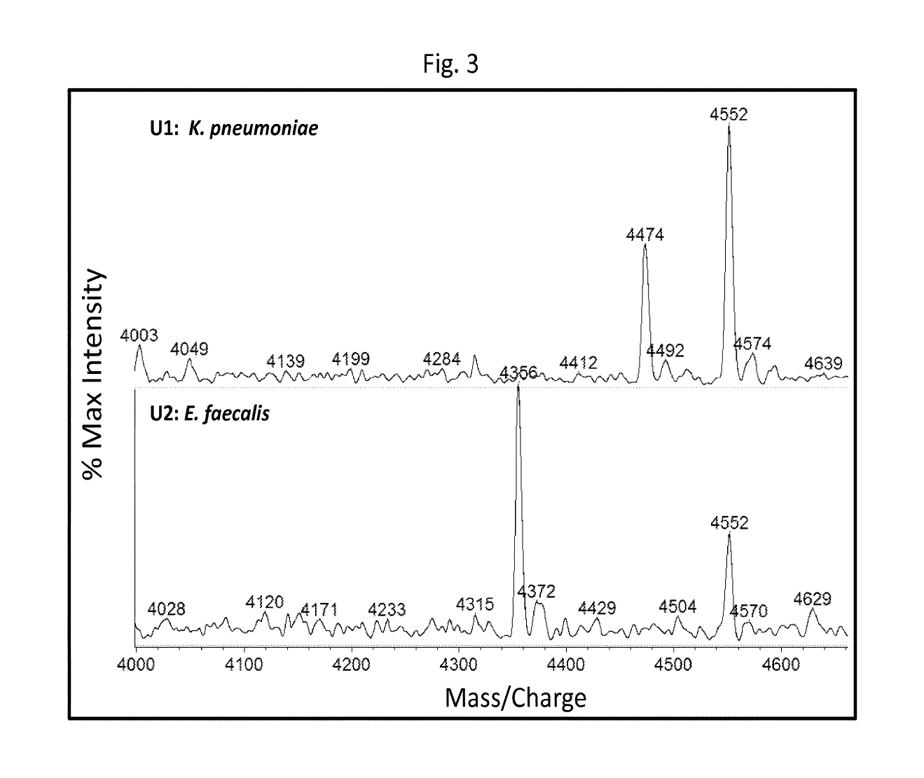

The data in Table 4 and FIG. 3 demonstrate that the method can be successfully applied to, not only TSB and blood cultures, but also primary (direct) urine samples. Two major peaks were recorded for each sample, one corresponding to the species-specific PNA probe and one corresponding to the universal bacterial probe.

### Example 13

### Detection of Two Bacterial Species in the Same Culture

The same Hybridization Solution as prepared according to Example 10 was applied to two (2) TSB cultures. Cultures of Staphylococcus aureus and Staphylococcus epidermidis were grown overnight, then mixed in a 0:1, 2:1, 1:2, 1:1, and 1:0 vol:vol ratio. Processing and hybridization was carried out as described under the heading: General Procedure for Detection of Microorganisms, except that the process was started at step 8. Identifications were made based on prominent peaks (those greater than approximately 10% of the maximum signal). Results are recorded in Table 5. The mass displayed in Table 5 “Peak 1” gave the greatest signal for each pair, followed by the mass displayed in “Peak 2,” followed by the mass displayed in “Peak 3.”

TABLE 5

| Ratio | | | | |
|---|---|---|---|---|
| S. aureus:S. | Peak | Peak | Peak | |
| epidermidis | 1 | 2 | 3 | MS Interpretation |
| 1:0 | 4507 | 4550 | N/A | S. aureus |
| 2:1 | 4508 | 4551 | 4097 | S. aureus |
| | | | | Non-aureus Staphylococci |
| 1:2 | 4508 | 4551 | 4096 | S. aureus |
| | | | | Non-aureus Staphylococci |
| 1:1 | 4551 | 4097 | 4508 | S. aureus |
| | | | | Non-aureus Staphylococci |
| 1:0 | 4096 | 4551 | N/A | Non-aureus Staphylococci |

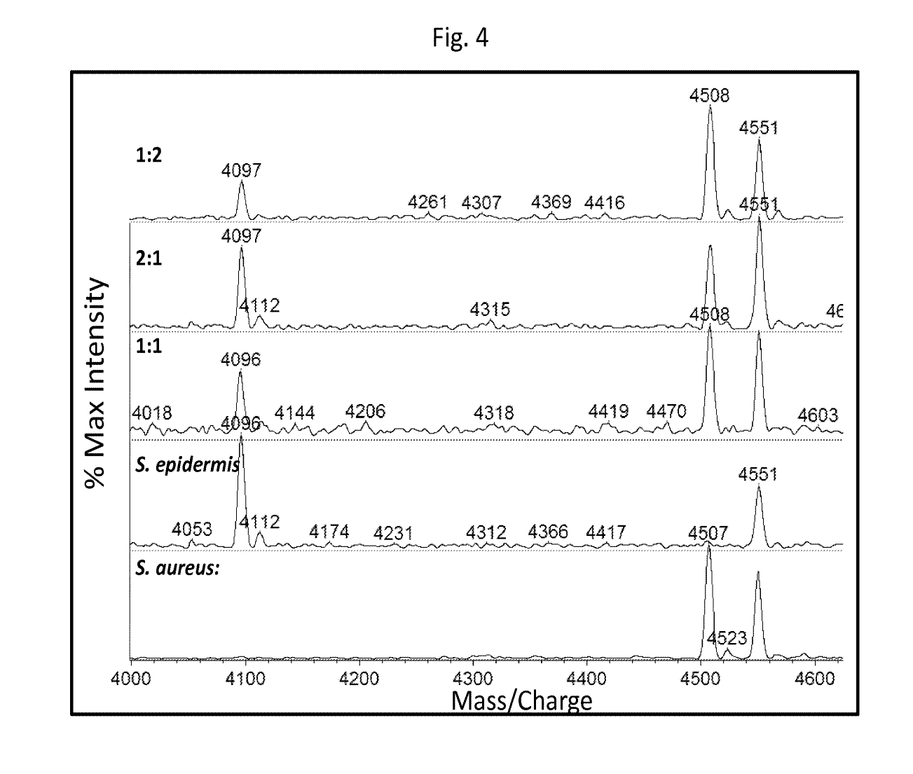

The data in Table 5 and FIG. 4 demonstrate that multiple microorganism can be detected simultaneously from one sample. All major peaks were recorded for each sample (peaks below approximately 10% maximum signal were ignored). One or two peaks corresponding to species specific PNA probes, and one corresponding to the universal bacterial probe were detected in all samples.

### Example 14

### Detection of Various Species of Bacteria Using a Smart Wash

Individual TSB cultures of five organisms including Staphylococcus epidermidis, Klebsiella pneumoniae, Staphylococcus aureus, Pseudomonas aeruginosa, and Escherichia coli were prepared in TSB. One milliliter of each culture was treated as described under heading: Smart Wash Procedure. The same Hybridization Solution as prepared according to Example 10, excluding probes B and C, was applied to each culture. Peaks below approximately 10% maximum signal were ignored. Table 6 identifies the dominant peaks observed for each sample.

TABLE 6

| | Peak | Peak | |
|---|---|---|---|
| Sample | 1 | 2 | MS Interpretation |
| Staphylococcus epidermidis | 4099 | 4553 | Non-aureus Staphylococci |
| Klebsiella pneumoniae | 4473 | 4551 | Klebsiella pneumoniae |
| Staphylococcus aureus | 4508 | 4551 | Staphylococcus aureus |
| Pseudomonas aeruginosa | 4552 | 4609 | Pseudomonas aeruginosa |
| Escherichia coli | 4552 | 4648 | Escherichia coli |

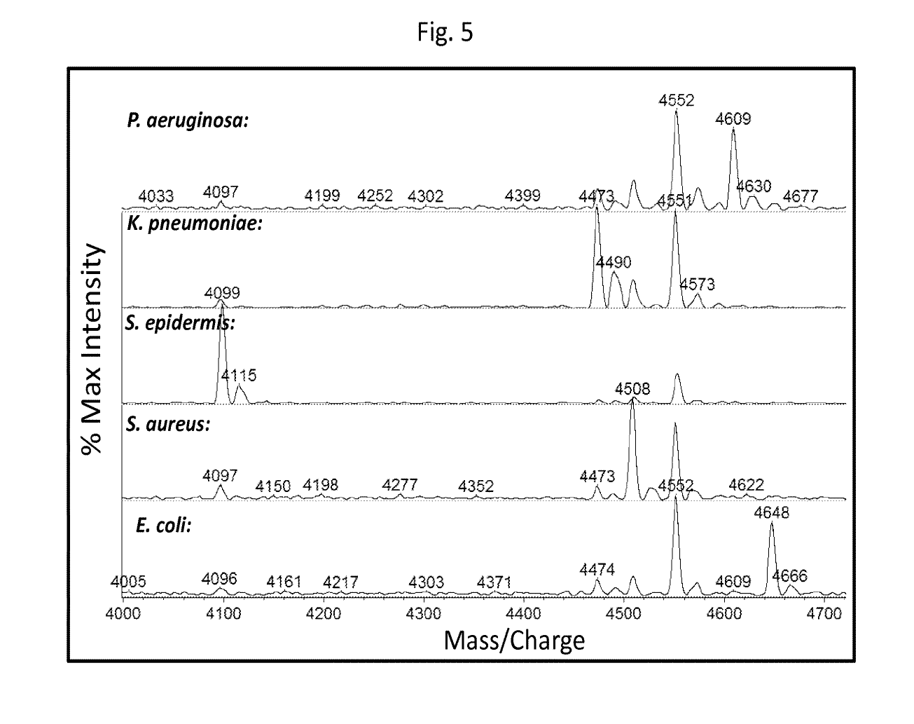

The data in Table 6 and FIG. 5 demonstrate that a streptavidin-coated solid support may be used to selectively remove non-hybridized hybridization probes from a solution of cells containing both hybridized and non-hybridized probes. The addition of this step saves time.

### Example 15

### Fluorescent Detection of Bacteria Using the Protocol Developed for MS

Individual Hybridization Solutions were prepared for each of two organisms, Staphylococcus aureus, and Escherichia coli. Each Hybridization Solution contained only one fluorescein-labeled, species-specific probe from Table 1 (either Probe of SEQ ID NO: 9, or Probe of SEQ ID NO: 10). These Hybridization Solutions were applied to their respective simulated blood culture. Five milliliters of each culture was treated as described under heading: General Procedure for Detection of Microorganisms except that after step 19, the pellet was resuspended in 50 μL Tris pH 9, and 5 μL of each sample was deposited onto a standard glass microscope slide. These samples were allowed to air dry, and then were mounted with standard fluorescence mounting media and a coverslip, and then examined on a fluorescent microscope, using a 60× oil objective and appropriate fluorescence filters.

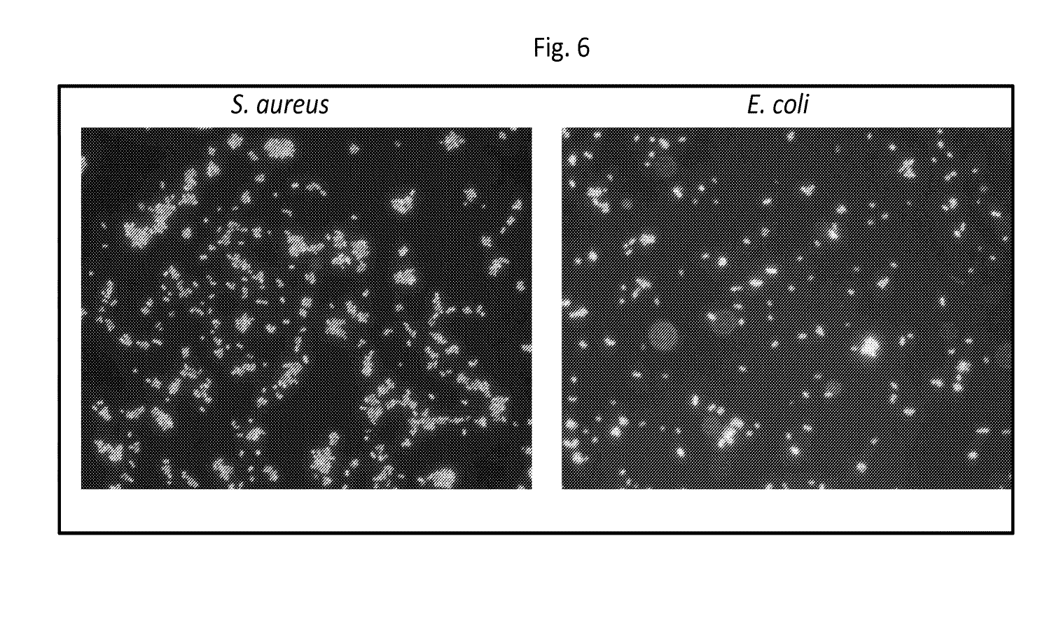

FIG. 6 shows the negative of fluorescence images taken of S. aureus and E. coli after specific hybridization. Negative controls (S. aureus cells hybridized with E. coli probe, and vice versa) are not displayed, but the cells did not produce any fluorescence above background. Images were processed to optimize contrast and simplify reproduction, but essentially demonstrate what could be seen by eye by the operator. The data in FIG. 6 demonstrate that after the cells are processed, and before they are flown on the mass spectrometer, the hybridization probes are located within the cells. These specific samples were not further processed on a mass spectrometer, but similar experiments have demonstrated the generation of m/z peaks associated with the mass of the fluorescein labeled probes.

All literature and similar materials cited in this application, including but not limited to patents, patent applications, articles, books and treatises, regardless of the format of such literature or similar material, are expressly incorporated by reference herein in their entirety for any and all purposes.

While the present teachings are described in conjunction with various embodiments, it is not intended that the present teachings be limited to such embodiments. On the contrary, the present teachings encompass various alternatives, modifications and equivalents, as will be appreciated by those of skill in the art. Thus, the invention as contemplated by applicants extends to all functionally equivalent structures, methods and uses, such as are within the scope of the appended claims.

Moreover, in the following claims it should be understood that the order of steps or order for performing certain actions (e.g., mixing of reactants) is immaterial so long as the present teachings remain operable. Unless expressly stated otherwise or where performing the steps of a claim in a certain order would be non-operative, the steps and/or substeps of the following claims can be executed in any order. Moreover, two or more steps or actions can be conducted simultaneously.

I/We claim:

1. A method comprising: 1) contacting microorganisms or cells of a sample with one or more hybridization probes for a period of time and under conditions sufficient for said hybridization probes to sequence specifically hybridize to their respective target sequences, if present, within said microorganisms or cells; 2) washing said microorganisms or cells to remove excess hybridization probes; and 3) analyzing said microorganisms or cells by mass spectrometry to identify one or more hybridization probes retained within said microorganisms or cells at a time after performing step 2.

2. The method of claim 1 further comprising lysing said microorganisms or cells at a time after performing step 2.

3. The method of claim 1 further comprising fixing said microorganisms or cells of said sample prior to performing step 1.

4. The method of claim 1, wherein said one or more hybridization probes hybridize to a target sequence associated with a condition of interest and said method further comprises determining one or more conditions of interest associated with said microorganisms or cells where said one or more conditions of interest correlates with said one or more hybridization probes identified in step 3) of the method.

5. The method of claim 4 further comprising making a diagnostic determination based upon said one or more conditions of interest so determined.

6. The method of claim 5 further comprising, making a treatment recommendation for a subject from whom said sample was obtained based upon said diagnostic determination.

7. The method of claim 1, wherein said one or more hybridization probes are PNA probes or PNA chimeras.

8. The method of claim 7, wherein the PNA probes or PNA chimeras comprise a formal positively charged label or a formal negatively charged label.

9. The method of claim 1, wherein said one or more hybridization probes comprise a signature mass tag.

10. The method of claim 1, wherein said one or more hybridization probes comprise a capture ligand.

11. The method of claim 10, wherein the washing step comprises contacting the sample with anti-ligand to said capture ligand wherein excess of said one or more hybridization probes is immobilized to a solid support and removed from said sample.

12. The method of claim 1, wherein said mass spectrometry is performed using a MALDI-TOF mass spectrometer.

13. The method of claim 4, wherein two or more hybridization probes are used and wherein at least two different hybridization probes each hybridize to a different complementary target sequence wherein each complementary target sequence correlates with a different condition of interest.

14. The method of claim 13, wherein each said different condition of interest is either a trait or the determination of a species, genus, class, order, family, phylum or other classification of said microorganism or cell.

15. The method of claim 4, wherein two or more hybridization probes are used and wherein at least two different hybridization probes each hybridize to a different complementary target sequence wherein each complementary target sequence correlates with the same condition of interest.

16. The method of claim 15, wherein each said condition of interest is either a trait or the determination of a species, genus, class, order, family, phylum or other classification of said microorganism or cell.

17. The method of claim 4, wherein said condition of interest is a trait.

18. The method of claim 4, wherein said condition of interest is the determination of a species, genus, class, order, family, phylum or other classification of said microorganism or cell.

19. The method of claim 2 further comprising concentrating the one or more hybridization probes released from the lysed microorganisms of cells and analyzing said one or more hybridization probes by mass spectrometry to thereby identify said released one or more hybridization probes.

20. The method of claim 17 wherein the trait is methicillin resistance.

21. The method of claim 1 wherein the sample is urine.

22. The method of claim 1 wherein the sample is a blood culture or a portion thereof.

23. The method of claim 1 wherein the sample is treated with a bioactive agent prior to performing, or during the performance of, step 1.

24. The method of claim 23, wherein the bioactive agent is an antibiotic.

25. The method of claim 1, wherein the target sequence is selected from the group consisting of mRNA, rRNA, plasmid DNA, viral nucleic acid and chromosomal DNA.

26. A method comprising: 1) contacting microorganisms or cells of a sample with one or more hybridization probes for a period of time and under conditions sufficient for said hybridization probes to sequence specifically hybridize to their respective target sequences, if present, within said microorganisms or cells; 2) washing said microorganisms or cells to remove excess hybridization probes; 3) treating said microorganisms or cells with heat and/or other denaturing conditions for a period of time sufficient to thereby cause said probe/target complexes to denature and said denatured hybridization probes to diffuse outside of the intact cells/microorganisms; 4) recovering said denatured hybridization probes that have diffused outside of said intact cells/microorganisms; and 5) analyzing said recovered denatured hybridization probes by mass spectrometry to thereby identify said recovered denatured hybridization probes.

27. The method of claim 26, wherein said one or more hybridization probes hybridize to a target sequence associated with a condition of interest and said method further comprises determining one or more conditions of interest associated with said microorganisms or cells where said one or more conditions of interest correlates with said one or more hybridization probes identified in step 5) of the method.

28. The method of claim 27 further comprising, making a diagnostic determination based upon said one or more conditions of interest so determined.

29. The method of claim 28 further comprising, making a treatment recommendation for a subject from whom said sample was obtained based upon said diagnostic determination.

30. A method comprising: 1) identifying one or more hybridization probes sequestered within cells or microorganisms by performing mass spectrometry on said cells or microorganisms; and 2) determining one or more conditions of interest associated with said cells or microorganisms based the so identified one or more hybridization probes.

31. The method of claim 30 further comprising making a diagnostic determination based upon said one or more conditions of interest so determined.

32. The method of claim 31 further comprising, making a treatment recommendation for a subject from whom said sample was obtained based upon said diagnostic determination.

33. The method of claim 32, wherein said one or more conditions of interest is a trait.

34. The method of claim 33, wherein the trait is methicillin resistance.

35. The method of claim 32, wherein said one or more conditions of interest is the determination of a species, genus, class, order, family, phylum or other classification of said microorganism or cell.

36. The method of claim 31, wherein said diagnostic determination is made based on the presence of one or more bacteria so determined as the one or more conditions of interest.

37. The method of claim 36, further comprising making a recommendation for treating a subject with an effective amount of an antibiotic known to effective towards said one or more bacteria so determined.


SEQUENCE LISTING (10 sequences)

SEQ ID NO: 1 - DNA, 13 bp, Artificial Sequence
  Labeled peptide nucleic acid sequence comprising an N-terminal Arginine and a C-terminal Lysine comprising a biotin labeled side chain.
agacgtgcatagt

SEQ ID NO: 2 - DNA, 14 bp, Artificial Sequence
  Labeled peptide nucleic acid sequence comprising an N-terminal Arginine and a C-terminal Lysine comprising a biotin labeled side chain
ccttctgatgggca

SEQ ID NO: 3 - DNA, 14 bp, Artificial Sequence
  Labeled peptide nucleic acid sequence comprising an N-terminal Arginine and a C-terminal Lysine comprising a biotin labeled side chain
cctctgatgggtag

SEQ ID NO: 4 - DNA, 15 bp, Artificial Sequence
  Labeled peptide nucleic acid sequence comprising an N-terminal Arginine and a C-terminal Lysine comprising a biotin labeled side chain
cacctacacaccagc

SEQ ID NO: 5 - DNA, 15 bp, Artificial Sequence
  Labeled peptide nucleic acid sequence comprising an N-terminal Arginine and a C-terminal Lysine comprising a biotin labeled side chain
gcttctcgtccgttc

SEQ ID NO: 6 - DNA, 15 bp, Artificial Sequence
  Labeled peptide nucleic acid sequence comprising an N-terminal Arginine and a C-terminal Lysine comprising a biotin labeled side chain
ctgcctcccgtagga

SEQ ID NO: 7 - DNA, 15 bp, Artificial Sequence
  Labeled peptide nucleic acid sequence comprising an N-terminal Arginine and a C-terminal Lysine comprising a biotin labeled side chain
ctgaatccaggagca

SEQ ID NO: 8 - DNA, 15 bp, Artificial Sequence
  Labeled peptide nucleic acid sequence comprising an N-terminal Arginine and a C-terminal Lysine comprising a biotin labeled side chain
tcaatgagcaaaggt

SEQ ID NO: 9 - DNA, 15 bp, Artificial Sequence
  Fluorescently labeled peptide nucleic acid sequence
gcttctcgtccgttc

SEQ ID NO: 10 - DNA, 15 bp, Artificial Sequence
  Fluorescently labeled peptide nucleic acid sequence
tcaatgagcaaaggt